(19) United States
(12) Patent Application Publication
(10) Pub. No.: US 2021/0046110 A1
(43) Pub. Date: Feb. 18, 2021

(54) MODIFIED MONOCYTES/MACROPHAGES/DENDRITIC CELLS EXPRESSING CHIMERIC ANTIGEN RECEPTORS AND USES IN DISEASES AND DISORDERS ASSOCIATED WITH PROTEIN AGGREGATES

(71) Applicant: THE TRUSTEES OF THE UNIVERSITY OF PENNSYLVANIA, Philadelphia, PA (US)

(72) Inventors: Saar Gill, Philadelphia, PA (US); Michael Klichinsky, Philadelphia, PA (US)

(21) Appl. No.: 16/965,568

(22) PCT Filed: Feb. 1, 2019
(86) PCT No.: PCT/US2019/016253

§ 371 Date: Jul. 28, 2020

Related U.S. Application Data
(60) Provisional application No. 62/625,487, filed on Feb. 2, 2018.
(60) Provisional application No. 62/786,875, filed on Dec. 31, 2018.

(51) Int. Cl. A61K 35/15; C07K 14/725; C12N 15/87

(52) U.S. Cl. CPC A61K 35/15; A61K 45/06; C12N 15/87; C07K 14/7051

(57) ABSTRACT

The present invention relates to compositions and methods for treating diseases and/or disorders associated with protein aggregates. By expressing a chimeric antigen receptor (CAR) in a monocyte, macrophage or dendritic cell, the modified cell is recruited or applied to the tissue microenvironment where it acts as a potent immune effector by infiltrating the tissue and eliminating, reducing, inhibiting or preventing protein aggregation. Other aspects of this invention include methods and pharmaceutical compositions comprising the CAR modified monocyte, macrophage or dendritic cell for treating a condition, such as a neurodegenerative disease/disorder, an inflammatory disease/disorder, a cardiovascular disease/disorder, a fibrotic disease/disorder and amyloidosis.

### CROSS REFERENCE TO RELATED APPLICATION

The present application is entitled to priority under 35 U.S.C. § 119(e) to U.S. Provisional Patent Application No. 62/625,487, filed Feb. 2, 2018 and U.S. Provisional Patent Application No. 62/786,875, filed Dec. 31, 2018, which are hereby incorporated by reference in their entireties herein.

### BACKGROUND OF THE INVENTION

A growing number of diseases and disorders are shown to be associated with inappropriate folding of proteins and/or inappropriate deposition and aggregation of proteins and lipoproteins as well as infectious proteinaceous substances. These include the well-established beta-amyloid and tau aggregates in Alzheimer's disease, alpha-synuclein aggregates in Parkinson's disease, FUS, TDP-43, OPTN, and C9ORF72 aggregation in conditions including amyotrophic lateral sclerosis (ALS) and the amyloid fibers and plaques characteristic of systemic amyloidosis. The pathogenic aggregation of proteins and/or lipoproteins not only occurs in neurodegenerative diseases but also in many other diseases such as inflammatory diseases, fibrotic diseases (e.g., collagen) and cardiovascular disease (e.g., LDL in atherosclerotic plaques).

Therapeutic approaches based on highly specific antibodies are currently being investigated for treating these diseases with the antibodies targeting different components of the pathological aggregate or directed to depleting a precursor protein for aggregate formation, thereby inhibiting or blocking aggregate formation. These antibodies can also be used to deplete aggregated proteins themselves, thereby inhibiting their spread to surrounding cells/tissues and/or blocking them seeding the formation of additional aggregates and/or preventing the exertion of their pathological effects. However, there are functional limitations of therapeutic antibodies, such as inadequate pharmacokinetics, failure to engage the cellular immune system, lack of retention or tissue penetration in target tissues (such as the blood brain barrier), off-site tissue toxicity, and lack of long-term maintenance effects in chronic disease conditions that still need to be addressed.

In patients with systemic amyloidosis, extracellular deposition of a normally intracellular protein leads to the accumulation of insoluble aggregates of amyloid (fibril sand the functional impairment of organs such as heart, liver, kidneys, nerves and blood vessels. Current treatments range from organ transplantation to chemotherapy that result in severe side effects and, in most cases, limited effectiveness. The introduction of monoclonal antibodies (mAbs) against the serum amyloid P component (SAP) leads to clearance of amyloid (Richards et al. N Engl J Med 2015; 373:1106-1114); however, the long-term maintenance of the deadly disease state remains unaddressed.

In models of prion disease, antibodies targeting the prion protein (PrP) lead to reduction in the insoluble pathogenic prion protein form PrP(Sc) and to reduction in brain lesions (Ohsawa et al, Microbiol Immunol. 2013 April; 57(4):288-97). However, due to the blood-brain barrier (BBB) and active transport out of the cerebrospinal fluid, brain mAb concentrations are 1000 times less than those in circulating blood, making it difficult to deliver mAbs to the brain at therapeutic levels.

In cardiovascular disease, a number of monoclonal antibody approaches have been taken, for example, the development of antibodies targeting the proprotein convertase subtilisin/kexin type 9 (PCSK9). However, the mechanism of action of anti PCSK9 antibodies lowers the levels of circulating low density lipoprotein (LDL) by making the LDL receptor more available to bind LDL and remove those particles from the circulation. These approaches do not target LDL particles and their removal directly, which would be helpful where LDL has already accumulated in the context of atherosclerotic plaque. Atherosclerotic lesions include various proteins and lipoproteins that can be targeted with antibodies and other targeting moieties, ideally through a drug substance that can penetrate the vascular endothelium/traffic into tissues and directly remove plaque build-up components. In addition, atherosclerotic plaques are known to be rich in macrophages, as macrophages serve as a precursor of foam cells. In atherosclerotic disease the vascular endothelium becomes more permeable to cells and monocytes easily penetrate into the intima, where they differentiate to macrophages and contribute to the formation of plaque (Lee et al., 2017. Lipids Health Dis. 2017; 16: 12).

The main treatment for atherosclerosis is lipid lowering, diabetes and hypertension control, and cessation of smoking. Patients with established atherosclerosis undergo stent insertion or bypass surgery. However, restenosis, and treatment side effects are common and there is a need to develop new therapeutic approaches for the treatment of atherosclerosis. The continued inflammatory environment of the atherosclerotic vessels, which attracts immune system cells, provides an opportunity to explore a therapy where a monocyte, macrophage or dendritic cell, drawn into a plaque but armed with the targeted ability to eliminate plaque components could have utility in directly reducing plaque burden in atherosclerosis.

Many chronic inflammatory diseases result in the development of fibrosis, a condition characterized with pathologic deposition of extracellular matrix components including collagen. Fibrosis affects different organs in the body notably the lungs (e.g., idiopathic pulmonary fibrosis) but also liver, kidney, heart, skin and others. The current treatments for fibrosis seek to manage the underlying inflammatory conditions but do not directly target the elimination of abnormal collagen deposits.

Therefore, a need exists for the development of new therapeutic modalities optimized to target specific antigens, proteins, glycoproteins or lipoproteins, particularly against diseases with pathologies based on protein misfolding and aggregation, as well as in cases of heterogeneous aggregates. The present invention addresses this need.

### SUMMARY

The present invention is based, at least in part, on the insight that cells and/or compositions comprising one or more antigen-binding domains may be useful in treating diseases, disorders, and/or conditions related to the formation of protein aggregates. The present invention is premised upon, among other things, the recognition that monocytes, macrophages, and/or dendritic cells, including monocytes, macrophages, and/or dendritic cells modified to express a chimeric antigen receptor or other antigen binding domain, may be used to disrupt protein aggregates found in a variety of diseases, disorders, and/or conditions. By way of non-limiting example, in some embodiments, disrupting a protein aggregate may be or comprise: reducing a previously formed protein aggregate in size, slowing or preventing the growth of a protein aggregate, and/or slowing or preventing the formation of a protein aggregate. Accordingly, the methods, cells, and compositions provided herein represent a powerful new class of therapeutics for a range of debilitating diseases, disorders, and/or conditions.

In some embodiments, provided cells and/or compositions may comprise an antigen binding domain that binds to a protein in or on a protein aggregate. In some embodiments, provided cells and/or compositions may comprise an antigen binding domain that binds to a structural epitope of a protein aggregate that includes portions of more than a single protein (e.g., a neoepitope). In some embodiments, provided cells and/or compositions may comprise an antigen binding domain that binds to a non-protein component of a protein aggregate.

In some embodiments, the present invention provides cells including a chimeric antigen receptor (CAR), wherein the CAR comprises an antigen binding domain, a transmembrane domain and an intracellular domain, wherein the antigen binding domain is capable of binding to an antigen of a protein aggregate, and wherein the cell is a monocyte, macrophage and/or a dendritic cell that expresses the CAR. In some embodiments, the present disclosure provides a CAR that includes one or more of: a linker/spacer domain, a co-stimulatory domain, and a destabilizing domain. In some embodiments, the present disclosure provides a cell, wherein the cell is a monocyte, macrophage and/or a dendritic cell expressing a CAR, wherein the cell also expresses one or more control systems selected from the group consisting of: a safety switch (e.g., an on switch, off switch, or suicide switch) and a logic gate (e.g., and AND, OR, or NOT gate).

In some embodiments, the present invention provides cells including an isolated nucleic acid sequence encoding a chimeric antigen receptor (CAR), wherein the isolated nucleic acid sequence comprises a nucleic acid sequence encoding an antigen binding domain, a nucleic acid sequence encoding a transmembrane domain and a nucleic acid sequence encoding an intracellular domain, wherein the antigen binding domain is capable of binding to an antigen of a protein aggregate, and wherein the cell is a monocyte, macrophage and/or a dendritic cell that expresses the CAR.

In accordance with several embodiments, any of a variety of antigen binding domains may be used. In some embodiments, an antigen binding domain is capable of binding to an antigen of a protein aggregate in a tissue of a subject with a neurodegenerative disease, an inflammatory disease, a cardiovascular disease, a fibrotic disease or amyloidosis. In some embodiments, an antigen binding domain is or comprises an antibody agent. In some embodiments, an antigen binding domain is or comprises an antibody agent selected from the group consisting of a monoclonal antibody, polyclonal antibody, synthetic antibody, human antibody, humanized antibody, single domain antibody, single chain variable fragment, and antigen-binding fragments thereof. In some embodiments, an antibody agent is or comprises a Tau antibody, a TDP-43 antibody, a beta-amyloid antibody, an amyloid antibody, a collagen antibody, and/or an scFv of any of the foregoing antibodies.

In accordance with various embodiments, any of a variety of intracellular domains may be used. In some embodiments, the intracellular domain is or comprises at least one of a co-stimulatory molecule and a signaling domain. In some embodiments, an intracellular domain of the CAR comprises dual signaling domains. In some embodiments, an intracellular domain of the CAR comprises more than two signaling domains. In some embodiments, an intracellular domain is from a co-stimulatory molecule selected from the group consisting of TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, and any combinations thereof. In some embodiments, an intracellular domain is or comprises CD3zeta. In some embodiments, an intracellular domain is or comprises FcERI, CD16, CD32, CD64, complement receptors, scavenger receptors, calreticulin receptors, ITGAM, SLAMF7, TREM2, Dectin-1, TLR1, 2, 3, 4, 5, 6, 7, 8, 9; MARCO, DAP12, MEGF10, CD19.

It is specifically contemplated that any of a variety of neurodegenerative diseases may be addressed (e.g., treated, cured, prevented, improved, and/or exhibit slowed progression) via use of certain embodiments. For example, in some embodiments, a neurodegenerative disease is selected from the group consisting of tauopathy, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, Familial British dementia, Fatal Familial Insomnia, Gerstmann-Straussler-Scheinker Syndrome, Hereditary cerebral hemorrhage with amyloidosis (Icelandic) (HCHWA-I), Sporadic Fatal Insomnia (sFI), Variably Protease-Sensitive Prionopathy (VPSPr), Familial Danish dementia, Creutzfeldt-Jakob disease (CJD), Variant Creutzfeldt-Jakob Disease (vCJD), and prion disease.

It is also contemplated that any of a variety of inflammatory diseases may be addressed (e.g., treated, cured, prevented, improved, and/or exhibit slowed progression) via use of certain embodiments. In some embodiments, an inflammatory disease is selected from the group consisting of systemic lupus erythematosus, vasculitis, rheumatoid arthritis, periodontitis, ulcerative colitis, sinusitis, asthma, tuberculosis, Crohn's disease, chronic infection, hereditary periodic fevers, malignancies, systemic vasculitides, diseases predisposing to recurrent infections, cystic fibrosis, bronchiectasis, epidermolysis bullosa, cyclic neutropenia, acquired or inherited immunodeficiencies, injection-drug use and acne conglobate, Muckle-Wells (MWS) disease and Familiar Mediterranean Fever (FMF), It is specifically contemplated that any of a variety of types of amyloidosis may be addressed (e.g., treated, cured, prevented, improved, and/or exhibit slowed progression) via use of certain embodiments. For example, in some embodiments, an amyloidosis is selected from the group consisting of Primary Amyloidosis (AL), Secondary Amyloidosis (AA), Familial Amyloidosis (ATTR), other Familial Amyloidoses, Beta-2 Microglobulin Amyloidosis, Localized Amyloidosis, Heavy Chain Amyloidosis (AH), Light Chain Amyloidosis (AL), Primary Systemic Amyloidosis, ApoAI Amyloidosis, ApoAII Amyloidosis, ApoAIV Amyloidosis, Apolipoprotein C2 Amyloidosis, Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis.

It is further contemplated that any of a variety of cardiovascular diseases may be addressed (e.g., treated, cured, prevented, improved, and/or exhibit slowed progression) via use of certain embodiments. In some embodiments, a cardiovascular disease is selected from the group consisting of atherosclerosis, coronary artery disease, peripheral artery disease, hypertensive heart disease, metabolic syndrome, hypertension, cerebrovascular disease, and heart failure.

It is further contemplated that any of a variety of fibrotic diseases may be addressed (e.g., treated, cured, prevented, improved, and/or exhibit slowed progression) via use of certain embodiments. In some embodiments, a fibrotic disease is selected from the group consisting of pulmonary fibrosis, idiopathic pulmonary fibrosis, cirrhosis, cystic fibrosis, scleroderma, cardiac fibrosis, radiation-induced lung injury, steatohepatitis, glomerulosclerosis, interstitial lung disease, liver fibrosis, mediastinal fibrosis, retroperitoneal cavity fibrosis, bone marrow fibrosis and skin fibrosis.

In accordance with several embodiments, provided cells and compositions may exhibit any of several beneficial activities (e.g., in a subject or patient). In some embodiments, a cell exhibits one or more activities selected from the group consisting of phagocytosis, targeted cellular cytotoxicity, antigen presentation, and cytokine secretion. In addition, in some embodiments, one or more activities of a provided cell may be enhanced or otherwise modulated using any of a variety of methods. By way of specific example, in some embodiments, an activity of a provided cell is enhanced by inhibition of CD47 and/or SIRPα activity.

It is specifically contemplated that, in some embodiments, provided cells and/or compositions may be used as a component of a combination therapy. In some embodiments, provided cell(s) and/or compositions may further include at least one agent selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate, a lipid, a hormone, a microsome, and any combinations thereof.

It is specifically contemplated that, in some embodiments, provided cells and/or compositions may be used in the manufacture of a medicament for the treatment of a neurodegenerative disease, an inflammatory disease, a cardiovascular disease, a fibrotic disease, or amyloidosis, in a subject in need thereof.

In accordance with various embodiments, the present invention provides a pharmaceutical composition comprising the provided cells and a pharmaceutically acceptable carrier. In some embodiments, the pharmaceutical composition further comprises at least one agent selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate, a lipid, a hormone, a microsome, and any combinations thereof.

In accordance with various embodiments, the present invention provides a method of treating a neurodegenerative disease, an inflammatory disease, a cardiovascular disease, a fibrotic disease, or amyloidosis, in a subject in need thereof, the method comprising administering to the subject a therapeutically effective amount of a pharmaceutical composition of the present invention.

In accordance with various embodiments, the present invention provides a method for stimulating an immune response to a target cell or tissue in a subject suffering from a neurodegenerative disease, an inflammatory disease, a cardiovascular disease, a fibrotic disease, or amyloidosis, the method comprising administering to a subject a therapeutically effective amount of a pharmaceutical composition of the present invention.

In accordance with various embodiments, the present invention provides a method of modifying a cell, the method comprising introducing into a monocyte, macrophage and/or dendritic cell a chimeric antigen receptor (CAR), wherein the CAR comprises an antigen binding domain, a transmembrane domain and an intracellular domain, wherein the antigen binding domain is or comprises an antibody agent capable of binding to an antigen of a protein aggregate. In some embodiments, introducing the CAR into the cell comprises introducing a nucleic acid sequence encoding the CAR into the cell. In some embodiments, introducing the nucleic acid sequence into the cell comprises electroporating an mRNA encoding the CAR into the cell. In some embodiments, introducing the nucleic acid sequence into the cell comprises at least one procedure selected from the group consisting of electroporation, a lentiviral transduction, adenoviral transduction, retroviral transduction and chemical-based transfection. In some embodiments, the method further comprises modifying the cell to deliver to a target an agent selected from the group consisting of a nucleic acid, an antibiotic, an anti-inflammatory agent, an antibody, a growth factor, a cytokine, an enzyme, a protein, a peptide, a fusion protein, a synthetic molecule, an organic molecule, a carbohydrate or the like, a lipid, a hormone, a microsome, and any combinations thereof. In some embodiments, the invention includes a composition comprising a cell made by a method of the present invention.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The foregoing and other features and advantages of the present invention will be more fully understood from the following detailed description of illustrative embodiments taken in conjunction with the accompanying drawings. It should be understood that the present invention is not limited to the precise arrangements and instrumentalities of the embodiments shown in the drawings.

FIG. 1 is a set of representative flow plots showing the expression of anti-amyloid CAR on THP1 macrophages.

FIG. 2 shows the elution profile of free light chains using a Sephadex 75 30/100 column (GE) connected to an AKTA purification system.

FIG. 3 shows protein fractions separated by size-exclusion chromatography analyzed by PAGE. Light chains free of heavy chains were identified in the 2nd and 3rd fractions.

FIG. 4 shows an electron micrograph of thermally denatured free light chains. EM negative staining shows long protein fibers.

FIG. 5 illustrates the uptake of fluorescently-labeled light chain amyloid fibrils by CAR-expressing macrophages. An increase in the MFI (mean fluorescence intensity) in the histograms on the bottom row of plots illustrates the uptake of amyloid fibrils.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. As used in this application, the terms “about” and “approximately” are used as equivalents. Any numerals used in this application with or without about/approximately are meant to cover any normal fluctuations appreciated by one of ordinary skill in the relevant art.

“Activation,” as used herein, refers to the state of a monocyte/macrophage/dendritic cell that has been sufficiently stimulated to induce detectable cellular proliferation or has been stimulated to exert its effector function. Activation can also be associated with induced cytokine production, phagocytosis, cell signaling, target cell killing, or antigen processing and presentation.

The term “activated monocytes/macrophages/dendritic cells” refers to, among other things, monocyte/macrophage/dendritic cells that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, and other functions.

The term “agent,” or “biological agent” or “therapeutic agent” as used herein, refers to a molecule that may be expressed, released, secreted or delivered to a target by the modified cell described herein. The agent includes, but is not limited to, a nucleic acid, an antibiotic, an anti-inflammatory agent, an antibody, antibody agent or fragments thereof, a growth factor, a cytokine, an enzyme, a protein, a peptide, a fusion protein, a synthetic molecule, an organic molecule (e.g., a small molecule), a carbohydrate or the like, a lipid, a hormone, a microsome, a derivative or a variation thereof, and any combinations thereof. The agent may bind any cell moiety, such as a receptor, an antigenic determinant, or other binding site present on a target or target cell. The agent may diffuse or be transported into the cell, where it may act intracellularly.

The term “antibody,” as used herein, refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

The term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is not and/or does not comprise a polypeptide whose amino acid sequence includes structural elements recognized by those skilled in the art as an immunoglobulin variable domain. In some embodiments, an antibody agent may be or comprise a molecule or composition which does not include immunoglobulin structural elements (e.g., a receptor or other naturally occurring molecule which includes at least one antigen binding domain).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that is capable of provoking an immune response. This immune response may involve either antibody production, or the activation of specific immunologically-competent cells, or both. The skilled artisan will understand that any macromolecule, including virtually all proteins or peptides, can serve as an antigen. Furthermore, antigens can be or be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “auto-antigen” means, in accordance with the present invention, any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, Epidermolysis Bullosa Simplex), epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “chimeric antigen receptor” or “CAR,” as used herein, refers to an artificial T cell surface receptor that is engineered to be expressed on an immune effector cell and specifically targets a cell and/or binds an antigen. CARs may be used as a therapy with adoptive cell transfer. Monocytes, macrophages and/or dendritic cells are removed from a patient (e.g., via blood or ascites fluid) and modified so that they express the receptors specific to a particular form of antigen. In some embodiments, the CARs have been expressed with specificity for amyloid protein antigens, for example. CARs may also comprise an intracellular activation domain, a transmembrane domain and an extracellular domain comprising, for example, an amyloid protein antigen binding region. In some aspects, CARs comprise fusions of single-chain variable fragments (scFv) derived monoclonal antibodies, CD3-zeta transmembrane domains and intracellular domains. The specificity of CAR designs may be derived from ligands of receptors (e.g., peptides). In some embodiments, a CAR can target a neurodegenerative, inflammatory, cardiovascular, fibritic or other disease/disorder by redirecting a monocyte, macrophage, or dendritic cell expressing the CAR specific for protein aggregates, associated with the disease/disorder.

The term “chimeric intracellular signaling molecule” refers to recombinant receptor comprising one or more intracellular domains of one or more stimulatory and/or co-stimulatory molecules. The chimeric intracellular signaling molecule substantially lacks an extracellular domain. In some embodiments, the chimeric intracellular signaling molecule comprises additional domains, such as a transmembrane domain, a detectable tag, and a spacer domain.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a monocyte/macrophage/dendritic cell, thereby providing a signal which mediates a monocyte/macrophage/dendritic cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a monocyte/macrophage/dendritic cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” or co-stimulatory domain” refers to a molecule on an innate immune cell that is used to heighten or dampen the initial stimulus. For example, pathogen-associated pattern recognition receptors, such as TLR (heighten) or the CD47/SIRPα axis (dampen), are molecules on innate immune cells. Co-stimulatory molecules include, but are not limited to TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combinations thereof.

A “co-stimulatory signal”, as used herein, refers to a signal, which in combination with a primary signal, such as activation of the CAR on a macrophage, leads to activation of the macrophage.

The term “cytotoxic” or “cytotoxicity” refers to killing or damaging cells. In one embodiment, cytotoxicity of the metabolically enhanced cells is improved, e.g. increased cytolytic activity of macrophages.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “neurodegenerative disease” as used herein, refers to a neurological disease characterized by loss or degeneration of neurons and/or by the presence of misfolded protein aggregates in the cytoplasm and/or nucleus of nerve cells or in the extracellular space (Forman et al., Nat. Med. 10, 1055 (2004)). Neurodegenerative diseases include neurodegenerative movement disorders and neurodegenerative conditions relating to memory loss and/or dementia. Neurodegenerative diseases include tauopathies and α-synucleopathies. Examples of neurodegenerative diseases include, but are not limited to, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS) and Hallervorden-Spatz syndrome.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of monocytes, macrophages, or dendritic cells. In one embodiment, the monocytes, macrophages, or dendritic cells that are expanded ex vivo increase in number relative to the number originally present in the culture. In another embodiment, the monocytes, macrophages, or dendritic cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses (e.g., Ad5F35), and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. As applied to the nucleic acid or protein, “homologous” as used herein refers to a sequence that has about 50% sequence identity. More preferably, the homologous sequence has about 75% sequence identity, even more preferably, has at least about 80%, 85%, 90%, 91%, 92%, 93%, 94%, 95%, 96%, 97%, 98%, 99% sequence identity.

“Humanized” forms of non-human (e.g., murine) antibodies are chimeric immunoglobulins, immunoglobulin chains or fragments thereof (such as Fv, scFv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The guide nucleic acid sequence may be complementary to one strand (nucleotide sequence) of a double stranded DNA target site. The percentage of complementation between the guide nucleic acid sequence and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The guide nucleic acid sequence can be at least 10, 11, 12, 13, 14, 15, 16, 17, 18, 19, 20, 21, 22, 23, 24, 25, 26, 27, 28, 29, 30, 31, 32, 33, 34, 35 or more nucleotides in length. In some embodiments, the guide nucleic acid sequence comprises a contiguous stretch of 10 to 40 nucleotides. The variable targeting domain can be composed of a DNA sequence, a RNA sequence, a modified DNA sequence, a modified RNA sequence (see for example modifications described herein), or any combinations thereof.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The terms “Lewy body”, “Lewy bodies”, and “Lewy neurites”, refer to abnormal aggregates of protein that develop in nerve cells.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), intratumoral (i.t.) or intra-peritoneal (i.p.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides.

As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combinations thereof.

The term “protein aggregate” as used herein means two or more proteins (e.g., two or more of the same protein, two or more different proteins, etc) that have aggregated together in a tissue in a subject to give rise to a pathological condition, or which places the subject at risk for a pathological condition. In some embodiments, a protein aggregate may be or comprise one or more of: misfolded protein(s), otherwise improperly formed/malformed protein(s) (e.g., as a result of a mutation which may not affect folding but does affect function), and/or an aggregation of protein and non-protein components (e.g., nucleic acids, small molecules, etc). Non-limiting examples of such protein aggregates include aggregates of amyloid protein, aggregates of tau protein, aggregates of TDP-43 protein, aggregates of immunoglobulin light chains or transthyretin protein, aggregates of prion protein and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “resistance to immunosuppression” refers to lack of suppression or reduced suppression of an immune system activity or activation.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

“Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, 1988, Science 242:423-442; Huston et al., 1988, Proc. Natl. Acad. Sci. USA 85:5879-5883; Ward et al., 1989, Nature 334:54454; Skerra et al., 1988, Science 242:1038-1041.

By the term “specifically binds,” as used herein with respect to an antigen binding domain, such as an antibody agent, is meant an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the Fc receptor machinery or via the synthetic CAR. Stimulation can mediate altered expression of certain molecules, such as downregulation of TGF-beta, and/or reorganization of cytoskeletal structures, and the like.

A “stimulatory molecule,” as the term is used herein, means a molecule of a monocyte, macrophage, or dendritic cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) or tumor cell can specifically bind with a cognate binding partner (referred to herein as a “stimulatory molecule”) on a monocyte, macrophage, or dendritic cell, thereby mediating a response by the immune cell, including, but not limited to, activation, initiation of an immune response, proliferation, and the like. Stimulatory ligands are well-known in the art and encompass, inter alia, Toll-like receptor (TLR) ligand, an anti-toll-like receptor antibody, an agonist, and an antibody for a monocyte/macrophage receptor. In addition, cytokines, such as interferon-gamma, are potent stimulants of macrophages.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

By “target” is meant a cell, organ, tissue or site within the body (e.g., a protein aggregate) that is in need of treatment.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory I′ cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention includes, inter alia, compositions and methods for treating a disease or disorder associated with protein aggregation in a subject. Diseases can include a neurodegenerative disease, an inflammatory disease, a cardiovascular disease, a fibrotic disease, amyloidosis, or any other disease or disorder with pathology based on the aggregation and/or misfolding of proteins or on the presence or activity of proteinaceous infectious particles. The invention includes expression of a chimeric antigen receptor (CAR) of any design in a monocyte, macrophage or dendritic cell. In some embodiments, such a modified cell is recruited or directly injected or applied to a diseased tissue microenvironment, where it acts as a potent immune effector by infiltrating and/or interacting with the tissue and modifying, neutralizing or eliminating a target protein/lipoprotein in a protein aggregate (including heterogeneous aggregates), misfolded protein, or infectious protein.

Among the advantages encompassed in the present disclosure is that use of cells such as monocytes, macrophages, and dendritic cells to express a CAR allows for the clearance of insoluble protein via the process of phagocytosis. Phagocytic uptake may lead to breakdown and digestion of pathogenic protein aggregates. Other cells, such as T cells and NK cells, do not have the capacity for phagocytosis. Without wishing to be held to a particular theory, among the advantages encompassed in the present disclosure is that use of cells such as monocytes, macrophages, and dendritic cells avoids the potential for the “cytokine storms” also referred to as “cytokine release syndrome” (CRS), which have proven to be a significant safety issue in traditional cell therapies (e.g., CAR-T).

In some embodiments, the present disclosure provides a cell (e.g., monocyte, macrophage or dendritic cell) comprising one or more control systems selected from the group consisting of: a safety switch (e.g., an on switch, off switch, or suicide switch) and a logic gate (e.g., and AND, OR, or NOT gate). In some embodiments, the present disclosure provides a cell (e.g., monocyte, macrophage or dendritic cell) comprising a “safety switch” (e.g., kill switch, suicide gene, on switch, off switch). In some embodiments, a safety switch comprises an enzyme involved in programmed cell death and a small molecule activator. In some embodiments, a safety switch comprises an enzyme involved in programmed cell death and an antibody activator. In some embodiments, the gene encoding the enzyme is transduced into a cell (e.g., monocyte, macrophage or dendritic cell) ex vivo. In some embodiments, activation of a safety switch leads to the death of the cell in which the safety switch has been activated. In some embodiments, activation of a safety switch leads to downregulation of the activity of the cell in which the safety switch has been activated, wherein the cell can be reactivated in the future. In some embodiments, the activity of the cell is downregulated by default and activation of a safety switch leads to upregulation of the activity of the cell, wherein the cell can be deactivated in the future.

Amyloid/Amyloidosis

Amyloidosis is term used to describe a group of diseases with the common pathological feature of abnormal build-up of incorrectly folded proteins known as amyloid or amyloid fibrils (Chiti and Dobson. 2006. Ann Review Biochem, 75: 333-366; Sipe et al., Amyloid 21(4): 221-224). An amyloid fibril is an insoluble protein complex deposited extracellularly as a result of misfolding of a soluble precursor protein (Nienhuis et al., Kidney Dis (Basel). 2016 April; 2(1): 10-19). The formation of the amyloid fibrils is the result of oligomerization and aggregation of the defective proteins. Amyloidosis can be localized or systemic and can be classified, according to one approach, in 6 groups: Primary Amyloidosis (AL), in which the amyloid fibrils are made up of immunoglobulin light chain proteins; Secondary Amyloidosis (AA), in which the source of amyloid is the serum amyloid A (SAA) as a result of inflammation; Familial Amyloidosis (ATTR), typically a result of a mutated transthyretin protein; Other Familial Amyloidoses with misfolding of different proteins leading to the disease pathology; Beta-2 Microglobulin Amyloidosis, where pathologic aggregates are made up of beta-2 microglobulin protein; and Localized Amyloidosis, associated with a variety of proteins in different tissues and organs (Boston University Amyloidosis Center).

Immunoglobulin light chain amyloidosis (AL) is a multisystem, lethal disorder characterized by organ dysfunction caused by the deposition of amyloid fibrils derived from an underlying plasma cell neoplasm. AL is a rare condition diagnosed in 3,000 patients in the US annually. The current treatment approach is chemotherapy directed to plasma cells and therefore is non-specific. The side-effects of this kind of treatment are associated with high early mortality (about one-third die within the first year) as well as delayed and incomplete clearance of the amyloid deposits from organs. This leaves patients with chronic morbidity from heart failure, nephrotic syndrome and disabling neuropathies along with ongoing risk of death. In order to improve outcomes in AL, therapies beyond the plasma cell are needed, in particular, clearance of deposited amyloid light chains. Herein, a treatment platform was developed based on macrophages expressing a chimeric antigen receptor (CAR macrophages) that identifies organ deposits of amyloid and clears the deposits through phagocytosis, thus leading to improvement in organ function, reduced morbidity and improved survival in AL. This treatment platform can be extended to additional types of amyloidosis as well as other diseases associated with extracellular deposits of misfolded protein.

Additional types of amyloidosis include but are not limited to Heavy Chain Amyloidosis (AH), primary systemic amyloidosis, ApoAI Amyloidosis, ApoAII Amyloidosis, ApoAIV Amyloidosis, Apolipoprotein C2 Amyloidosis, and Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis.

Examples of additional amyloid-associated disease include: Alzheimer's disease, where an aggregation of Tau protein and beta-amyloid is observed; spongiform encephalopathies (prion diseases), where mutated prion proteins make up the toxic aggregates; cataracts, caused by aggregation of the protein crystallin; type 2 diabetes, with aggregates made from amylin and others (Caughey and Lansbury. 2003. Annu Rev Neurosci. 26:267-98; Valastyan and Lindquist, Disease Models & Mechanisms (2014) 7, 9-14).

Proteins implicated in amyloidosis include, but are not limited to, serum amyloid A (SAA) protein, monoclonal immunoglobulin light proteins (kappa or lambda), immunoglobulin heavy chain proteins, transthyretin protein, apolipoprotein A-I (AApoAI), apolipoprotein A-II (AApoAII), apolipoprotein A-IV (AApoAIV), apolipoprotein C2 (ApoC2), apolipoprotein C3 (ApoC3), keratin, amyloid Dan (ADan), lactoferrin, gelsolin (AGel or GSN), fibrinogen (AFib), fibrinogen alpha chain (FGA), lysozyme (ALys or LYZ), Lect2, beta-2 microglobulin, amyloid beta, crystallin, amylin (islet amyloid peptide), prion protein (PrP), leukocyte cell derived chemotaxin 2 (LECT2), cystatin C (CST3), oncostatin M receptor (OSMR), integral membrane protein 2B (ITM2b), prolactin (PRL), keratoepithelin and atrial natriuretic factor (ANF). Amyloidosis may present via any of a variety of signs or symptoms including, but not limited to, swelling of extremities, especially ankles and/or legs, fatigue, shortness of breath, weight loss, irregular heartbeat, numbness in hands or feet, tingling or pain in hands or feet, and/or shortness of breath. These clinical manifestations reflect involvement of most major organ systems but particularly heart, kidneys, and peripheral nerves.

In some embodiments, provided compositions may be used with one or more other treatments for amyloidosis including, but not limited to chemotherapy, stem cell therapy, anti-inflammatory agents, or myeloma-directed therapy such as proteasome inhibitors among others.

The compositions and methods disclosed herein may comprise an antibody, antibody agent, and/or other antigen binding domain against common epitopes of amyloid aggregates or epitopes that are distinct for different misfolded proteins contributing to the formation of an amyloid fibril, herein referred to as “amyloid”. The term amyloid includes naturally occurring human amino acid sequences both wild type and mutated as well as fragments, analogs including allelic, species and induced variants. Amino acids of analogs are assigned the same numbers as corresponding amino acids in the natural human sequence when the analog and human sequence are maximally aligned. Analogs typically differ from naturally occurring peptides at one, two or a few positions, often by virtue of conservative substitutions. The term “allelic variant” is used to refer to variations between genes of different individuals in the same species and corresponding variations in proteins encoded by the genes. Anti-amyloid antibodies, their fragments, and analogs can be synthesized by solid phase peptide synthesis or recombinant expression, or can be obtained from natural sources. Automatic peptide synthesizers are commercially available from numerous suppliers, such as Applied Biosystems, Foster City, Calif.

### Amyloid Beta/Alzheimer's Disease

Alzheimer's Disease (AD) is a type of progressive dementia, characterized by worsening memory loss over time. The disease is the sixth leading cause of death in the US, and there is no current therapy to treat the underlying pathology of AD. The main pathology observed in the brains of AD patients is the accumulation of extracellular aggregates/plaques, composed of amyloid beta protein, including Aβ₄₂ (Sadigh-Eteghad et al. 2014. Medical Principles and Practice. 24 (1): 1-10). Beta amyloid protein can form plaques in other disease conditions such as other dementias (Lewy body) or muscle diseases.

While AD sufferers may exhibit any of a variety of signs or symptoms, common signs or symptoms include loss of memory (e.g., short term memory or long-term memory), inhibition of reasoning capacity, inhibition or loss of ability to make decisions, impaired planning ability, changes to personality, and/or altered behavior patterns (e.g., depression, mood swings, loss of inhibition, apathy, and withdrawal).

The symptoms of AD worsen over time, although the rate at which the disease progresses varies. On average, a subject with AD lives four to eight years after diagnosis, but can live as long as 20 years, depending on other factors. Changes in the brain related to AD begin years before any signs of the disease. This time period, which can last for years, is referred to as preclinical AD.

A subject in the early stage of AD (mild AD) may function independently, for example by driving, working and participating in social activities. In some embodiments, a subject suffering from early AD may feel as if he or she is having memory lapses, such as forgetting familiar words or the location of everyday objects. In some embodiments, friends, family or others close to a subject with early AD begin to notice difficulties. In some embodiments, a doctor performing a detailed medical interview with a subject suffering from early AD may be able to detect problems in memory or concentration. In some embodiments, a subject suffering from early stage AD experiences one or more difficulties selected from a group consisting of: problems coming up with the right word or name, trouble remembering names when introduced to new people, challenges performing tasks in social or work settings, forgetting material that one has just read, losing or misplacing a valuable object, and increasing trouble with planning or organizing.

The middle stage of AD (moderate AD) is typically the longest stage and can last for many years. As the disease progresses, a subject with AD will require a greater level of care. In some embodiments, a subject suffering from the middle stage of AD can experience symptoms selected from the group consisting of: confusing words, getting frustrated or angry, and acting in unexpected ways (e.g., refusing to bathe or other personality changes). Neurodegeneration in the brain of a subject with moderate AD can make it difficult for the subject to express thoughts and perform routine tasks. In some embodiments, symptoms in a subject suffering from the middle stage of AD will be noticeable to others outside of close family. In some embodiments, a subject suffering from the middle stage of AD experiences one or more difficulties selected from a group consisting of: and may include: forgetfulness of events or about the subject's own personal history, feeling moody or withdrawn, especially in socially or mentally challenging situations, being unable to recall the subject's own address or telephone number or the high school or college from which the subject graduated, confusion about where the subject is or what day it is, the need for help choosing proper clothing for the season or the occasion, trouble controlling bladder and bowels, changes in sleep patterns (e.g., sleeping during the day and becoming restless at night), an increased risk of wandering and becoming lost, personality and behavioral changes (e.g., suspiciousness and delusions) and compulsive, repetitive behavior (e.g., hand-wringing or tissue shredding).

In the final stage of AD (severe AD), a subject loses the ability to respond to his or her environment, to carry on a conversation and, eventually, to control movement. In some embodiments, a subject suffering from the final stage of AD may still say words or phrases, but communicating pain becomes difficult. In some embodiments, a subject suffering from the final stage of AD experiences significant personality changes. In some embodiments, a subject suffering from the final stage of AD needs extensive help with daily activities. In some embodiments, a subject suffering from the final stage of AD experiences one or more difficulties selected from a group consisting of: requiring round-the-clock assistance with daily activities and personal care, losing awareness of recent experiences as well as of surroundings, experiencing changes in physical abilities (e.g., the ability to walk, sit and, eventually, swallow), having increasing difficulty communicating, and becoming vulnerable to infections (e.g., pneumonia).

In some embodiments, a composition according to the present invention is administered to a subject suffering from the early stage of AD. In some embodiments, a composition according to the present invention is administered to a subject suffering from the middle stage of AD. In some embodiments, a composition according to the present invention is administered to a subject suffering from the final stage of AD.

Current treatments for AD include acetyl cholinesterase inhibitors and the N-methyl-D-aspartate receptor antagonist Memantine, but offer symptomatic rather than disease-modifying benefits (Malik and Robertson. 2017. J Neurol 264:416-418). In some embodiments, the present invention comprises treating a subject suffering from AD with a composition comprising modified cells comprising chimeric antigen receptors (CARs) as described herein. In some embodiments, treating a subject suffering from AD comprises administering a CAR-based therapeutic composition as described herein alone or in combination with an additional (non-CAR) therapeutic composition. In some embodiments, treating a subject suffering from AD with only a CAR-based therapeutic composition as described herein has a greater effect on the AD symptoms and/or pathology of the subject than treating a subject suffering from AD with only the additional (non-CAR) therapeutic composition. In some embodiments, treating a subject suffering from AD with a combination of a CAR-based therapeutic composition as described herein and an additional therapeutic composition has a synergistic effect on the AD symptoms and/or pathology of the subject. In some embodiments, the additional therapeutic composition comprises a human monoclonal antibody that selectively targets aggregated Aβ. In some embodiments, the human monoclonal antibody that selectively targets aggregated Aβ is aducanumab. In some embodiments, the additional therapeutic composition comprises a selective inhibitor of tau protein aggregation. In some embodiments, the selective inhibitor of tau protein aggregation is Leuco-methylthioninium bishydromethanesulfonate (LM™).

In some embodiments, the effect of an AD treatment on AD symptoms in a subject is evaluated using the Alzheimer's Disease Assessment Scale-Cognitive Subscale (ADAS-Cog) and/or the AD Co-operative Study-Activities of Daily Living Inventory (ADCS-ADL). In some embodiments, the effect of an AD treatment on AD pathology in a subject is evaluated by measuring the level of protein aggregates in the brain of the subject. In some embodiments, protein aggregates are aggregated Aβ(e.g., Aβ₄₂). In some embodiments, protein aggregates are tau protein aggregates.

### Collagen/Fibrotic Diseases

Abnormal deposition of collagen occurs in both systemic disorders such as systemic sclerosis (scleroderma) and chronic graft-versus-host disease, as well as in organ-specific disorders such as various pulmonary fibrotic disorders and hepatic cirrhosis.

Systemic Sclerosis

Systemic sclerosis (SSc) is a connective tissue disease (CTD), which affects skin, blood vessels, heart, lungs, kidneys, gastrointestinal (GI) tract and musculoskeletal system and includes development of collagen aggregates as a characterizing feature. Involvement of internal organs results in significant morbidity and mortality of patients with SSc (Kowal-Bielecka O, et al. Ann Rheum Dis 2017; 76:1327-1339). In some embodiments, symptoms of SSc comprise a hardening and tightening of patches of skin. In some embodiments, symptoms of SSc comprise an exaggerated response to cold temperatures or emotional distress, which can cause numbness, pain or color changes in the fingers or toes (also referred to as “Raynaud's phenomenon”). In some embodiments, symptoms of SSc comprise acid reflux and/or problems absorbing nutrients (e.g., if intestinal muscles aren't moving food properly through the intestines). In some embodiments, symptoms of SSc comprise abnormal function of the heart, lungs or kidneys to varying degrees.

Current therapies for Raynaud's phenomenon (RP) in subjects with systemic sclerosis include dihydropyridine-type calcium antagonists (e.g., nifedipine), PDE-5 inhibitors, prostanoids (e.g., intravenous iloprost) and fluoxetine. Current therapies for digital ulcers in subjects with systemic sclerosis include intravenous iloprost, PDE-5 inhibitors, and Bosentan. Current therapies for pulmonary arterial hypertension (PAH) include endothelin receptor antagonists (e.g., ambrisentan, bosentan and macitentan), PDE-5 inhibitors (e.g., sildenafil and tadalafil) and riociguat. Current therapies for skin and lung issues in subjects with systemic sclerosis include methotrexate, cyclophosphamide, and hematopoietic stem cell transplantation (HSCT). Current therapies for scleroderma renal crisis in subjects with systemic sclerosis include ACE inhibitors. Current therapies for SSc-related gastrointestinal disease include proton pump inhibitors, prokinetic drugs and intermittent or rotating antibiotics (to treat symptomatic small intestine bacterial overgrowth).

In some embodiments, the present invention provides methods including a step of treating a subject suffering from SSc with a composition comprising modified cells comprising chimeric antigen receptors (CARs) as described herein. In some embodiments, treating a subject suffering from SSc comprises administering a CAR-based therapeutic composition as described herein alone or in combination with an additional (non-CAR) therapeutic composition. In some embodiments, treating a subject suffering from SSc with only a CAR-based therapeutic composition as described herein has a greater effect on the SSc symptoms and/or pathology of the subject than treating a subject suffering from SSc with only the additional (non-CAR) therapeutic composition. In some embodiments, treating a subject suffering from SSc with a combination of a CAR-based therapeutic composition as described herein and an additional therapeutic composition has a synergistic effect on the SSc symptoms and/or pathology of the subject.

Graft v Host Disease

Chronic graft-versus-host disease (GVHD) is the most serious and common long-term complication of allogeneic hematopoietic stem cell transplantation (HSCT), occurring in 20% to 70% of people surviving more than 100 days (Lee, S. J. Blood. 2005 Jun. 1; 105(11): 4200-4206). Approximately half of affected people have 3 or more involved organs, and treatment typically requires immunosuppressive medications for a median of 1 to 3 years. Despite the well-recognized adverse effects of chronic GVHD on the long-term success of allogeneic transplantation, its pathophysiology is poorly understood, and management strategies beyond systemic corticosteroids have not been established.

In some embodiments, signs and symptoms of chronic GVHD include: joint or muscle pain, shortness of breath, persistent cough, vision changes (e.g., dry eyes), skin changes (e.g., scarring under the skin or skin stiffness), rash, yellow tint to skin or the whites of the eyes (jaundice), dry mouth, mouth sores, abdominal pain, diarrhea, nausea, and vomiting.

Treatments aimed at target tissues may minimize morbidity and improve functionality in subjects suffering from chronic GVHD. One of the major debilitating tissue responses is fibrosis. Halofuginone has been given topically or systemically to inhibit TGF-β-induced collagen α1 gene overexpression. Halofuginone inhibits smad3 phosphorylation, particularly in fibroblasts induced to oversecrete collagen by activation with TGF-β or activating mutations, via a mechanism that relies on protein synthesis. Excess collagen deposition may also be combated through physical rehabilitation, similar to the treatment of burn victims and people with scleroderma, many of whom also suffer from excess collagen deposition. Aggressive heat therapy, massage, and passive range-of-motion exercises can help maintain function until the sclerotic process can be controlled.

In some embodiments, the present invention provides methods including a step of treating a subject suffering from chronic GVHD with a composition comprising modified cells comprising chimeric antigen receptors (CARs) as described herein. In some embodiments, treating a subject suffering from chronic GVHD comprises administering a CAR-based therapeutic composition as described herein alone or in combination with an additional (non-CAR) therapeutic composition. In some embodiments, treating a subject suffering from chronic GVHD with only a CAR-based therapeutic composition as described herein has a greater effect on the chronic GVHD symptoms and/or pathology of the subject than treating a subject suffering from chronic GVHD with only the additional (non-CAR) therapeutic composition. In some embodiments, treating a subject suffering from chronic GVHD with a combination of a CAR-based therapeutic composition as described herein and an additional therapeutic composition has a synergistic effect on the chronic GVHD symptoms and/or pathology of the subject. In some embodiments, the additional (non-CAR) therapeutic composition comprises Halofuginone.

Pulmonary Fibrosis

Pulmonary fibrosis (PF) is a lung disease that occurs when lung tissue becomes damaged and scarred. This thickened, stiff tissue makes it more difficult for the lungs to work properly. As pulmonary fibrosis worsens, shortness of breath becomes progressively worse. The scarring associated with pulmonary fibrosis can be caused by a multitude of factors, but in most cases, doctors can't determine the cause of PF and when a cause can't be found, the condition is termed idiopathic pulmonary fibrosis. In some embodiments, signs and symptoms of PF include: shortness of breath (dyspnea), a dry cough, fatigue, unexplained weight loss, aching muscles and joints, and widening and rounding of the tips of the fingers or toes (clubbing).

Current therapies for PF include nintedanib (Ofev, a tyrosine kinase inhibitor that targets multiple tyrosine kinases, including vascular endothelial growth factor, fibroblast growth factor, and PDGF receptors) and pirfenidone (Esbriet, a pyridone that reduces fibroblast proliferation, inhibits TGF-β stimulated collagen production, and reduces the production of fibrogenic mediators such as TGF-β).

In some embodiments, the present invention provides methods including a step of treating a subject suffering from PF with a composition comprising modified cells comprising chimeric antigen receptors (CARs) as described herein. In some embodiments, treating a subject suffering from PF comprises administering a CAR-based therapeutic composition as described herein, alone or in combination with an additional (non-CAR) therapeutic composition. In some embodiments, treating a subject suffering from PF with only a CAR-based therapeutic composition as described herein has a greater effect on the PF symptoms and/or pathology of the subject than treating a subject suffering from PF with only the additional (non-CAR) therapeutic composition. In some embodiments, treating a subject suffering from PF with a combination of a CAR-based therapeutic composition as described herein and an additional therapeutic composition has a synergistic effect on the PF symptoms and/or pathology of the subject. In some embodiments, the additional (non-CAR) therapeutic composition comprises nintedanib. In some embodiments, the additional (non-CAR) therapeutic composition comprises pirfenidone.

Hepatic Cirrhosis

Hepatic cirrhosis is a late stage of hepatic fibrosis that has resulted in widespread distortion of normal hepatic architecture. In some embodiments, cirrhosis is characterized by regenerative nodules surrounded by dense fibrotic tissue. In some embodiments, signs and symptoms of cirrhosis include: fatigue, bleeding easily, bruising easily, itchy skin, jaundice, ascites (fluid accumulation in the abdomen), loss of appetite, nausea, swelling in the legs, weight loss, hepatic encephalopathy (confusion, drowsiness and slurred speech), spiderlike blood vessels on the skin, redness in the palms of the hands, testicular atrophy and breast enlargement (in men).

Current therapies for cirrhosis are supportive in nature and include stopping injurious drugs, providing nutrition and treating underlying disorders and complications. Liver transplantation is indicated for patients with end-stage liver disease.

In some embodiments, the present invention provides methods including a step of treating a subject suffering from cirrhosis with a composition comprising modified cells comprising chimeric antigen receptors (CARs) as described herein. In some embodiments, treating a subject suffering from cirrhosis comprises administering a CAR-based therapeutic composition as described herein, alone or in combination with an additional (non-CAR) therapeutic composition. In some embodiments, treating a subject suffering from cirrhosis with only a CAR-based therapeutic composition as described herein has a greater effect on the cirrhosis symptoms and/or pathology of the subject than treating a subject suffering from cirrhosis with only the additional (non-CAR) therapeutic composition. In some embodiments, treating a subject suffering from cirrhosis with a combination of a CAR-based therapeutic composition as described herein and an additional therapeutic composition has a synergistic effect on the cirrhosis symptoms and/or pathology of the subject.

### Lipoproteins/Cardiovascular Disease

Atherosclerosis is a vascular disease in which heterogeneous plaques composed of lipids, lipoproteins, proteins and other substances build up in the wall of the arteries, obstructing the lumen of the vessel, and often leading to pain and tissue damage. The main component of atherosclerotic plaque is low density lipoprotein (LDL), which is thought to initiate the formation of plaques by entering the lining of the blood vessels through a damaged epithelial cell layer. The epithelium could be damaged by smoking, diabetes or other conditions. The accumulation of LDL leads to an inflammatory reaction that attracts monocytes (eventually turning into foam cells), and the catalysis of plaque growth.

In some cases, the plaque can “rupture” and the resulting clot could cause a myocardial infarction or stroke. The three main diseases caused by atherosclerotic plaque are coronary artery disease (when the plaque is in the coronary arteries and can lead to chest pain or angina), cerebrovascular disease (when the atherosclerotic plaque is in the brain, and can cause transient ischemic attacks), and peripheral artery disease (leads to poor circulation in the extremities with pain, difficulty walking, poor wound healing, and, in extreme cases, the need for amputation).

Atherosclerosis is a chronic disease, which develops over many years with co-morbidities necessitating daily maintenance with pharmaceuticals, which target reduction in LDL, thinning of the blood, lowering of blood pressure and others but do not directly target dissolution of plaque. Years of taking maintenance medications for atherosclerosis can result in damage to the liver. In addition to medicines, atherosclerosis can be treated with medical procedures such as reopening of the vessel with or without stenting (percutaneous coronary intervention or PCI), coronary artery bypass grafting (CABG) surgery, bypass grafting in the extremities or carotid endarterectomy.

Additional indications that can be treated with the subject CAR of the present invention include, but are not limited to, cerebral autosomal dominant arteriopathy with subcortical infarcts and leukoencephalopathy (CADASIL), Cerebral β-amyloid angiopathy, Phenylketonuria (PKU), Pulmonary alveolar proteinosis (autoimmune), and Pulmonary alveolar proteinosis (congenital).

### Chimeric Antigen Receptor (CAR)

In one aspect of the invention, a modified monocyte, macrophage, or dendritic cell is generated by expressing a CAR therein. Thus, the present invention encompasses provided CARs, as well as nucleic acid constructs encoding provided CARs, wherein the CAR includes an antigen binding domain, a transmembrane domain and an intracellular domain. In certain instances, a monocyte, macrophage or dendritic cell comprising a CAR is referred to herein as a CAR-Macrophage.

In one aspect, the invention includes a cell including a chimeric antigen receptor (CAR), wherein the CAR comprises an antigen binding domain, a transmembrane domain and an intracellular domain, wherein the antigen binding domain is capable of binding to an antigen of a protein aggregate, and wherein the cell is a monocyte, macrophage and/or a dendritic cell that expresses the CAR.

In another aspect, the present invention provides cells including a nucleic acid sequence (e.g., an isolated nucleic acid sequence) encoding a chimeric antigen receptor (CAR), wherein the nucleic acid sequence comprises a nucleic acid sequence encoding an antigen binding domain, a nucleic acid sequence encoding a transmembrane domain and a nucleic acid sequence encoding an intracellular domain, wherein the antigen binding domain is capable of binding to an antigen of a protein aggregate, and wherein the cell is a monocyte, macrophage and/or a dendritic cell that expresses the CAR. In some embodiments, a single nucleic acid sequence may encode at least two of an antigen binding domain, a transmembrane domain, and an intracellular domain.

In one aspect, the invention includes a modified cell comprising a chimeric antigen receptor (CAR), wherein the CAR comprises an antigen binding domain, a transmembrane domain and an intracellular domain of a co-stimulatory molecule, wherein the antigen binding domain comprises an antibody agent or fragment thereof that is capable of binding to a protein in a protein aggregate in a tissue of a subject with a neurodegenerative disease, an inflammatory disease, a cardiovascular disease, a fibrotic disease, or amyloidosis, and wherein the modified cell is a monocyte, macrophage, or dendritic cell that possesses targeted effector activity. In another aspect, the invention includes a modified cell comprising a nucleic acid sequence encoding a chimeric antigen receptor (CAR), wherein the nucleic acid sequence comprises a nucleic acid sequence encoding an antigen binding domain, a nucleic acid sequence encoding a transmembrane domain and a nucleic acid sequence encoding an intracellular domain of a co-stimulatory molecule, wherein the nucleic acid sequence encoding the antigen binding domain comprises an antibody or fragment thereof that is capable of binding to a protein in a protein aggregate in a tissue of a subject with a neurodegenerative disease, an inflammatory disease, a cardiovascular disease, a fibrotic disease or amyloidosis, and wherein the cell is a monocyte, macrophage, or dendritic cell that expresses the CAR and possesses targeted effector activity. In one embodiment, the targeted effector activity is directed against an antigen on a target cell that specifically binds the antigen binding domain of the CAR. In another embodiment, the targeted effector activity is selected from the group consisting of phagocytosis, targeted cellular cytotoxicity, antigen presentation, and cytokine secretion.

Antigen Binding Domain

In some embodiments, a provided CAR comprises one or more antigen binding domains that bind to an antigen of a protein aggregate and/or an antigen on the surface of a target cell. Examples of cell surface markers that may act as an antigen that binds to the antigen binding domain of the CAR include those associated with viral, bacterial and parasitic infections, autoimmune disease/disorder, neurodegenerative disease/disorder, inflammatory disease/disorder, cardiovascular disease/disorder, fibrotic disease/disorder, and amyloidosis.

The choice of antigen binding domain depends upon the type and number of antigens that are present in a protein aggregate or on the surface of a target cell. For example, the antigen binding domain may be chosen to recognize an antigen that acts as a cell surface marker on a target cell associated with a particular disease or disorder state.

In some embodiments, an antigen binding domain binds to a misfolded protein antigen or a protein of a protein aggregate, such as a protein that is specific for a disease/disorder of interest. In some embodiments, the disease/disorder is a neurodegenerative disease/disorder, an inflammatory disease/disorder, a cardiovascular disease/disorder, a fibrotic disease/disorder, or amyloidosis (e.g., mediated by protein aggregates of immunoglobulin light chains or of transthyretin). In some embodiments, the neurodegenerative disease/disorder is selected from the group consisting of tauopathy, α-synucleopathy, presenile dementia, senile dementia, Alzheimer's disease (mediated by protein aggregates of beta-amyloid), Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down syndrome, multiple system atrophy, amyotrophic lateral sclerosis (ALS), Hallervorden-Spatz syndrome, polyglutamine disease, trinucleotide repeat disease, Familial British dementia, Fatal Familial Insomnia, Gerstmann-Straussler-Scheinker Syndrome, Hereditary cerebral hemorrhage with amyloidosis (Icelandic) (HCHWA-I), Sporadic Fatal Insomnia (sF1), Variably Protease-Sensitive Prionopathy (VPSPr), Familial Danish dementia, and prion disease (such as Creutzfeldt-Jakob disease, CJD and Variant Creutzfeldt-Jakob Disease (vCJD)).

The antigen binding domain can include any domain that binds to the antigen and may be or include, but is not limited to, a monoclonal antibody, a polyclonal antibody, a synthetic antibody, a human antibody, a humanized antibody, a non-human antibody, and any fragment thereof, for example a scFv. In addition, in some embodiments, an antigen binding domain can be or include an aptamer, a darpin, a naturally occurring or synthetic receptor, affibodies, or other engineered protein recognition molecule. Thus, in some embodiments, an antigen binding domain portion comprises a mammalian antibody or a fragment thereof. In another embodiment, the antigen binding domain of the CAR is selected from the group consisting of an anti-Tau antibody, an anti-TDP-43 antibody, an anti-beta-amyloid antibody, an anti-amyloid antibody and an anti-collagen antibody, or fragment thereof (e.g., an scFV).

In some instances, an antigen binding domain is derived, in whole or in part, from the same species in which the CAR will ultimately be used in. For example, for use in humans, in some embodiments, an antigen binding domain of the CAR may be or comprise a human antibody, a humanized antibody, or a fragment thereof.

In some aspects of the invention, an antigen binding domain is operably linked to another domain of a provided CAR, such as the transmembrane domain or the intracellular domain, for expression in the cell. In some embodiments, a nucleic acid encoding an antigen binding domain is operably linked to a nucleic acid encoding a transmembrane domain and the transmembrane domain is operably linked to a nucleic acid encoding an intracellular domain. In some embodiments, a modified cell (e.g., a modified monocyte, macrophage, or dendritic cell) comprising a CAR further comprises an additional antigen-binding domain that is required for activation (e.g., a bispecific CAR or bispecific modified cell). In some embodiments, a bispecific modified cell can reduce off-target and/or on-target off-tissue effects by requiring that two antigens are present. In some embodiments, a CAR and an additional antigen-binding domain provide distinct signals that in isolation are insufficient to mediate activation of the modified cell, but are synergistic together, stimulating activation of the modified cell. In some embodiments, such a construct may be referred to as an ‘AND’ logic gate.

In some embodiments, a bispecific modified cell can reduce off-target and/or on-target off-tissue effects by requiring that one antigen is present (e.g., a misfolded protein antigen or a protein of a protein aggregate) and a second, normal protein antigen is absent before the cell's activity is stimulated. In some embodiments, such a construct may be referred to as a ‘NOT’ logic gate. In contrast to AND gates, NOT gated CAR-modified cells are activated by binding to a single antigen. However, the binding of a second receptor to the second antigen functions to override the activating signal being perpetuated through the CAR. This inhibitory receptor would be targeted against an antigen that is abundantly expressed in a normal tissue but is absent in misfolded proteins or protein aggregates.

Transmembrane Domain

With respect to the transmembrane domain, a provided CAR can be designed to comprise a transmembrane domain that connects the antigen binding domain of the CAR to the intracellular domain. In some embodiments, a transmembrane domain may be naturally associated with one or more of the domains in a CAR. In some instances, a transmembrane domain can be selected or modified by amino acid substitution to avoid binding of such domain(s) to the transmembrane domains of the same or different surface membrane proteins to minimize interactions with other members of the receptor complex.

In some embodiments, a transmembrane domain may be derived either from a natural or from a synthetic source. Where the source is natural, the domain may be derived from any membrane-bound or transmembrane protein. In some embodiments, transmembrane regions of particular use may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some embodiments, a transmembrane region may comprise one or more hinge regions. In some instances, any of a variety of human hinge regions can be employed as well (e.g., a CD28 or CD8 hinge region) including the human Ig (immunoglobulin) hinge region.

In some embodiments, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain.

Intracellular Domain

In some embodiments, an intracellular domain or other cytoplasmic domain of the CAR may be or include a similar or the same intracellular domain as the chimeric intracellular signaling molecule described elsewhere herein, and is responsible for activation of the cell in which the CAR is expressed.

In some embodiments, an intracellular domain of a CAR includes a domain responsible for signal activation and/or transduction.

Examples of an intracellular domain for use in some embodiments include, but are not limited to, the cytoplasmic portion of a surface receptor, co-stimulatory molecule, and any molecule that acts in concert to initiate signal transduction in a monocyte, macrophage or dendritic cell, as well as any derivative or variant of these elements and any synthetic sequence that has the same functional capability.

Examples of intracellular domains useful in some embodiments, include those that comprise a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combinations thereof.

In some embodiments, an intracellular domain of a CAR comprises dual signaling domains, such as 41BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, an intracellular domain of a CAR includes any portion of one or more co-stimulatory molecules, such as at least one signaling domain from CD3, Fc epsilon RI gamma chain, any derivative or variant thereof, any synthetic sequence thereof that has the same functional capability, and any combinations thereof.

In some embodiments, between the antigen binding domain and the transmembrane domain of a provided CAR, or between the intracellular domain and the transmembrane domain of a provided CAR, a spacer domain may be incorporated. As used herein, the term “spacer domain” generally means any oligo- or polypeptide that functions to link the transmembrane domain to either the antigen binding domain or to the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Human Antibodies

In some embodiments, it may be preferable to use human antibodies or fragments thereof as an antigen binding domain of a CAR. Completely human antibodies are particularly desirable for therapeutic treatment of human subjects. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, it has been described that the homozygous deletion of the antibody heavy chain joining region (JH) gene in chimeric and germ-line mutant mice results in complete inhibition of endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Antibodies directed against the target of choice can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies, including, but not limited to, IgG1 (gamma 1) and IgG3. For an overview of this technology for producing human antibodies, see, Lonberg and Huszar (Int. Rev. Immunol., 13:65-93 (1995)). For a detailed discussion of this technology for producing human antibodies and human monoclonal antibodies and protocols for producing such antibodies, see, e.g., PCT Publication Nos. WO 98/24893, WO 96/34096, and WO 96/33735; and U.S. Pat. Nos. 5,413,923; 5,625,126; 5,633,425; 5,569,825; 5,661,016; 5,545,806; 5,814,318; and 5,939,598, each of which is incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above. For a specific discussion of transfer of a human germ-line immunoglobulin gene array in germ-line mutant mice that will result in the production of human antibodies upon antigen challenge see, e.g., Jakobovits et al., 1993, Proc. Natl. Acad. Sci. USA, 90:2551; Jakobovits et al., 1993, Nature, 362:255-258; Bruggermann et al., 1993, Year in Immunol., 7:33; and Duchosal et al., 1992, Nature, 355:258.

Human antibodies can also be derived from phage-display libraries (Hoogenboom et al., J. Mol. Biol., 227:381 (1991); Marks et al., J. Mol. Biol., 222:581-597 (1991); Vaughan et al., Nature Biotech., 14:309 (1996)). Phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of unimmunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905, each of which is incorporated herein by reference in its entirety.

Human antibodies may also be generated by in vitro activated B cells (see, U.S. Pat. Nos. 5,567,610 and 5,229,275, each of which is incorporated herein by reference in its entirety). Human antibodies may also be generated in vitro using hybridoma techniques such as, but not limited to, that described by Roder et al. (Methods Enzymol., 121:140-167 (1986)).

### Humanized Antibodies

In some embodiments, a non-human antibody can be humanized, where specific sequences or regions of the antibody are modified to increase similarity to an antibody naturally produced in a human. For instance, in some embodiments, an antibody or fragment thereof may comprise a non-human mammalian scFv. In some embodiments, an antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies. Humanization of antibodies can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making humanized antibodies is typically made to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993), the contents of which are incorporated herein by reference herein in their entirety).

Antibodies can be humanized that retain high affinity for a target antigen and that possess other favorable biological properties. According to one aspect of the invention, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind the target antigen. In this way, FR residues can be selected and combined from the recipient and import sequences so that the desired antibody characteristic, such as increased affinity for the target antigen, is achieved. In general, the CDR residues are directly and most substantially involved in influencing antigen binding.

In some embodiments, a humanized antibody retains a similar antigenic specificity as the original antibody. However, using certain methods of humanization, the affinity and/or specificity of binding of the antibody to the target antigen may be increased using methods of “directed evolution,” as described by Wu et al., J. Mol. Biol., 294:151 (1999), the contents of which are incorporated herein by reference herein in their entirety.

### Vectors

In some embodiments, a vector may be used to introduce a CAR into a monocyte, macrophage or dendritic cell as described elsewhere herein. In one aspect, the invention includes a vector comprising a nucleic acid sequence encoding a CAR as described herein. In some embodiments, the vector comprises a plasmid vector, viral vector, retrotransposon (e.g. piggyback, sleeping beauty), site directed insertion vector (e.g. CRISPR, Zn finger nucleases, TALEN), or suicide expression vector, or other known vector in the art.

In some embodiments, constructs mentioned above are capable of use with 3rd generation lentiviral vector plasmids, other viral vectors, or RNA approved for use in human cells. In some embodiments, the vector is a viral vector, such as a lentiviral vector. In some embodiments, the vector is a RNA vector.

The production of any of the molecules described herein can be verified by sequencing. Expression of the full length proteins may be verified using immunoblot, immunohistochemistry, flow cytometry or other technology well known and available in the art.

In some embodiments, the present invention also provides vectors in which DNA of the present invention is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

The expression of natural or synthetic nucleic acids is typically achieved by operably linking a nucleic acid or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

In accordance with various embodiments, a nucleic acid can be cloned into any number of different types of vectors. For example, in some embodiments, a nucleic acid can be cloned into a vector including, but not limited to a plasmid, a phagemid, a phage derivative, an animal virus, and a cosmid. Vectors of particular interest in some embodiments include expression vectors, replication vectors, probe generation vectors, and sequencing vectors.

In some embodiments, an expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193, the contents of which are incorporated herein by reference in their entireties).

Additional promoter elements, e.g., enhancers, regulate the frequency of transcriptional initiation and may be useful in some embodiments. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used in accordance with various embodiments, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-la promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of a reporter gene is assessed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82, which is incorporated herein by reference in its entirety). Suitable expression systems are well known and may be prepared using known techniques or obtained commercially. In general, a construct with the minimal 5′ flanking region showing the highest level of expression of reporter gene is identified as the promoter. Such promoter regions may be linked to a reporter gene and used to evaluate agents for the ability to modulate promoter-driven transcription.

### Introduction of Nucleic Acids

In some embodiments, the invention includes methods for modifying cells comprising introducing a nucleic acid sequence encoding some or all of a chimeric antigen receptor (CAR) into a monocyte, macrophage or dendritic cell, wherein the CAR comprises an antigen binding domain, a transmembrane domain and an intracellular domain, wherein the antigen binding domain is capable of binding to an antigen of a protein aggregate, and wherein the cell is a monocyte, macrophage and/or a dendritic cell that expresses the CAR.

In some embodiments, the invention includes methods for modifying a cell comprising introducing a nucleic acid sequence (e.g., an isolated or non-native nucleic acid sequence) encoding a chimeric antigen receptor (CAR) into a monocyte, macrophage or dendritic cell, wherein the isolated nucleic acid sequence comprises a nucleic acid sequence encoding an antigen binding domain, a nucleic acid sequence encoding a transmembrane domain and a nucleic acid sequence encoding an intracellular domain, wherein the antigen binding domain is capable of binding to an antigen of a protein aggregate, and wherein the cell is a monocyte, macrophage and/or a dendritic cell that expresses the CAR. In some embodiments, one or more of the antigen binding domain, transmembrane domain, and the intracellular domain are encoded by separate nucleic acid molecules.

In some embodiments, the invention includes methods for modifying a cell comprising introducing a chimeric antigen receptor (CAR) into the monocyte, macrophage, or dendritic cell, wherein the CAR comprises an antigen binding domain, a transmembrane domain and an intracellular domain, wherein the antigen binding domain, e.g., an antibody, antibody agent, etc., is capable of binding to a protein aggregate in a tissue of a subject with a neurodegenerative disease/disorder, an inflammatory disease/disorder, a cardiovascular disease/disorder, a fibrotic disease/disorder or amyloidosis, and wherein the cell is a monocyte, macrophage, or dendritic cell that expresses the CAR and possesses targeted effector activity. In some embodiments, introducing the CAR into the cell comprises introducing a nucleic acid sequence encoding the CAR (e.g., some components or all of the CAR). In some embodiments, introducing the nucleic acid sequence comprises electroporating DNA or a mRNA encoding the CAR into a cell.

Methods of introducing and expressing genes, such as those that encode a CAR, into a cell are known in the art. In the context of an expression vector, the vector can be readily introduced into a host cell, e.g., mammalian, bacterial, yeast, or insect cell by any method in the art. For example, in some embodiments, an expression vector can be transferred into a host cell by physical, chemical, or biological means.

Physical methods for introducing a polynucleotide into a host cell include calcium phosphate precipitation, lipofection, particle bombardment, microinjection, electroporation, squeeze technology, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection, using lipofection, using polymer encapsulation, using peptide mediated transfection, or using biolistic particle delivery systems such as “gene guns” (see, for example, Nishikawa, et al. Hum Gene Ther., 12(8):861-70 (2001).

In some embodiments, biological methods for introducing a polynucleotide of interest into a host cell may be or include the use of DNA and RNA vectors. RNA vectors include vectors having a RNA promoter and/or other relevant domains for production of a RNA transcript. Viral vectors, and especially retroviral vectors, have become the most widely used method for inserting genes into mammalian, e.g., human, cells. Other viral vectors may be derived from lentivirus, poxviruses, herpes simplex virus, adenoviruses (e.g. Ad5F35) and adeno-associated viruses, and the like. See, for example, U.S. Pat. Nos. 5,350,674 and 5,585,362.

In some embodiments, the present invention provides methods of modifying a cell, the method comprising introducing into a monocyte, macrophage and/or dendritic cell a nucleic acid encoding a chimeric antigen receptor (CAR), wherein the CAR comprises an antigen binding domain, a transmembrane domain and an intracellular domain, wherein the antigen binding domain is or comprises an antibody agent capable of binding to an antigen of a protein aggregate. In some embodiments, introducing a nucleic acid sequence into the cell comprises adenoviral transduction. In some embodiments, adenoviral transduction comprises use of an Ad5F35 adenovirus vector. In some embodiments, an Ad5F35 adenovirus vector is a helper-dependent Ad5F35 adenovirus vector. In some embodiments, an AD5F35 adenovirus vector is an integrating, CD46-targeted, helper-dependent adenovirus HDAd5/35++ vector system.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, where a non-viral delivery system is utilized, an exemplary delivery vehicle may be or comprise a liposome. The use of lipid formulations is contemplated for the introduction of the nucleic acids into a host cell (in vitro, ex vivo or in vivo). In another aspect, a nucleic acid may be associated with a lipid. In some embodiments, a nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, one or more nucleic acid sequences are introduced by a method selected from the group consisting of transducing the population of cells, transfecting the population of cells, and electroporating the population of cells. In some embodiments, a population of cells comprises one or more of the nucleic acid sequences described herein. In some embodiments, one or more nucleic acids are transfected, transduced and/or electroporated with one or more nuclease enzymes (e.g. Cas9 or Cas12a, for example).

In some embodiments, nucleic acids introduced into the cell are or comprise RNA. In some embodiments, RNA is mRNA that comprises in vitro transcribed RNA or synthetic RNA. In some embodiments, RNA is produced by in vitro transcription using a polymerase chain reaction (PCR)-generated template. DNA of interest from any source can be directly converted by PCR into a template for in vitro mRNA synthesis using appropriate primers and RNA polymerase. In some embodiments, the source of the DNA can be, for example, genomic DNA, plasmid DNA, phage DNA, cDNA, synthetic DNA sequence or any other appropriate source of DNA. In some embodiments, a desired template for in vitro transcription is or comprises a CAR.

In some embodiments, PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

Some in vitro-transcribed RNA (IVT-RNA) vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In some embodiments, an RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of Cells

In some embodiments, phagocytic cells are used in the compositions and methods described herein. In some embodiments, a source of phagocytic cells, such as monocytes, macrophages and/or dendritic cells, is obtained from a subject. Non-limiting examples of subjects include humans, dogs, cats, mice, rats, and transgenic species thereof. Preferably, the subject is a human. In some embodiments, cells can be obtained from a number of sources, including peripheral blood mononuclear cells, bone marrow, lymph node tissue, spleen tissue, umbilical cord, and induced pluripotent stem cells. In certain embodiments, any number of monocyte, macrophage, dendritic cell or progenitor cell lines available in the art, may be used. In certain embodiments, cells can be obtained from a unit of blood collected from a subject using any number of techniques known to the skilled artisan, such as Ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, precursors to monocytes, macrophages, or dendritic cells may be used (e.g., stem cells). Non-limiting examples include, hematopoietic stem cells, common myeloid progenitors, myeloblasts, monoblasts, promonocytes, and intermediates. In another embodiment, induced pluripotent stem cells may be used as a source of generating monocytes, macrophages, and/or dendritic cells.

If myeloid precursors are used, such as hematopoietic stem cells, they may be ex vivo differentiated into monocytes, macrophages, and/or dendritic cells, or precursors of said pathway. In addition, precursors (such as but not limited to hematopoietic stem cells) may be used as the therapeutic cell, such that the myeloid differentiation occurs in vivo. Cells may be autologous or sourced from allogeneic or universal donors. In some embodiments, myeloid progenitors or hematopoietic stem cells may be engineered such that expression of the CAR is under the control of a cell type specific promoter, such as a known myeloid, macrophage, monocyte, dendritic cell, microglial cell, M1 specific, or M2 specific promoter.

In some embodiments, monocytes or precursors may be ex vivo differentiated into microglial cells prior to infusion with cytokines known to those in the art. In some embodiments, differentiation of monocytes into microglial cells may improve activity in the central nervous system.

In some embodiments, induced pluripotent stem cells may be derived from normal human tissue, such as peripheral blood, fibroblasts, skin, keratinocytes, renal epithelial cells, or other cells reprogrammed with the genes OCT4, SOX2, KLF4, and C-MYC. In some embodiments, autologous, allogeneic, or universal donor iPSCs could be differentiated toward the myeloid lineage (monocyte, macrophage, dendritic cell, and/or precursor thereof).

In some embodiments, cells are isolated from peripheral blood by lysing the red blood cells and depleting the lymphocytes and red blood cells, for example, by centrifugation through a PERCOLL™ gradient. Alternatively, cells can be isolated from umbilical cord. In any event, a specific subpopulation of the monocytes, macrophages and/or dendritic cells can be further isolated by positive or negative selection techniques.

The mononuclear cells so isolated can be depleted of cells expressing certain antigens, including, but not limited to, CD34, CD3, CD4, CD8, CD14, CD19 or CD20. Depletion of these cells can be accomplished using an isolated antibody, a biological sample comprising an antibody, such as ascites fluid, an antibody bound to a physical support, and a cell bound antibody.

Enrichment of a monocyte, macrophage and/or dendritic cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, enrich of a cell population for monocytes, macrophages and/or dendritic cells by negative selection can be accomplished using a monoclonal antibody cocktail that typically includes antibodies to CD34, CD3, CD4, CD8, CD14, CD19 or CD20.

During isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. The use of high concentrations of cells can result in increased cell yield, cell activation, and cell expansion.

In some embodiments, a population of cells comprises the monocytes, macrophages, or dendritic cells of the present invention. Examples of a population of cells include, but are not limited to, peripheral blood mononuclear cells, cord blood cells, a purified population of monocytes, macrophages, or dendritic cells, and a cell line. In some embodiments, peripheral blood mononuclear cells comprise the population of monocytes, macrophages, or dendritic cells. In some embodiments, purified cells comprise the population of monocytes, macrophages, or dendritic cells.

In some embodiments, cells may have upregulated M1 markers and/or downregulated M2 markers. For example, in some embodiments, at least one M1 marker, such as HLA DR, CD86, CD80, and PDL1, is upregulated in the phagocytic cell. In another example, in some embodiments, at least one M2 marker, such as CD206, CD163, is downregulated in the phagocytic cell. In one embodiment, the cell has at least one upregulated M1 marker and at least one downregulated M2 marker.

In some embodiments, targeted effector activity in the phagocytic cell is enhanced by inhibition of either CD47 or SIRPα activity. CD47 and/or SIRPα activity may be inhibited by treating the phagocytic cell with an anti-CD47 or anti-SIRPα antibody. Alternatively, CD47 or SIRPα activity may be inhibited by any method known to those skilled in the art.

### Expansion of Cells

In some embodiments, cells or population of cells comprising monocytes, macrophages, or dendritic cells are cultured for expansion. In some embodiments, cells or population of cells comprising progenitor cells are cultured for differentiation and expansion of monocytes, macrophages, or dendritic cells. In some embodiments, the present invention comprises expanding a population of monocytes, macrophages, or dendritic cells comprising a chimeric antigen receptor as described herein.

As demonstrated by the data disclosed herein, expanding the cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the cells expand in the range of about 20 fold to about 50 fold.

Following culturing, cells can be incubated in cell medium in a culture apparatus for a period of time or until the cells reach confluency or high cell density for optimal passage before passing the cells to another culture apparatus. In some embodiments, a culturing apparatus can be any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The culture medium may be replaced during the culture of the cells at any time. Preferably, the culture medium is replaced about every 2 to 3 days. The cells are then harvested from the culture apparatus whereupon the cells can be used immediately or stored for use at a later time

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

In some embodiments, cells may be cultured for several hours (about 3 hours) to about 14 days or any hourly integer value in between. Conditions appropriate for cell culture include an appropriate media (e.g., macrophage complete medium, DMEM/F12, DMEM/F12-10 (Invitrogen)) that may contain factors necessary for proliferation and viability, including serum (e.g., fetal bovine or human serum), L-glutamine, insulin, M-CSF, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of the cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the cells may include an agent that can activate the cells. For example, an agent that is known in the art to activate the monocyte, macrophage or dendritic cell is included in the culture medium.

### Therapy

In some embodiments, modified cells described herein may be included in a composition for treatment of a subject. In one aspect, the composition comprises the modified cell comprising the chimeric antigen receptor described herein. In some embodiments, a provided composition may include a pharmaceutical composition and further include a pharmaceutically acceptable carrier. In some embodiments, a therapeutically effective amount of the pharmaceutical composition comprising the modified cells may be administered.

In one aspect, the invention provides methods of treating a disease/disorder or condition associated with a neurodegenerative disease/disorder, an inflammatory disease/disorder, a cardiovascular disease/disorder, a fibrotic disease/disorder or amyloidosis in a subject comprising administering to the subject a therapeutically effective amount of a pharmaceutical composition comprising modified cells as described herein. In another aspect, the invention provides methods for stimulating an immune response to a target a diseased/disordered cell or tissue in a subject comprising administering to a subject a therapeutically effective amount of a pharmaceutical composition comprising modified cells as described herein. In yet another aspect, the invention includes use of provided modified cells as described herein in the manufacture of a medicament for the treatment of an immune response in a subject in need thereof. In still another aspect, the invention includes use of provided modified cells as described herein in the manufacture of a medicament for the treatment of a neurodegenerative disease/disorder, an inflammatory disease/disorder, a cardiovascular disease/disorder, a fibrotic disease/disorder or amyloidosis in a subject in need thereof.

In some embodiments, provided modified cells generated as described herein possess targeted effector activity. In some embodiments, provided modified cells have targeted effector activity directed against an antigen on a target cell, such as through specific binding to an antigen binding domain of a CAR. In some embodiments, targeted effector activity includes, but is not limited to, phagocytosis, targeted cellular cytotoxicity, antigen presentation, and cytokine secretion.

In some embodiments, modified cells described herein have the capacity to deliver an agent, for example, a biological agent or a therapeutic agent to the target. In some embodiments, a cell may be modified or engineered to deliver an agent to a target, wherein the agent is selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate or the like, a lipid, a hormone, a microsome, a derivative or a variation thereof, and any combinations thereof. As a non-limiting example, a macrophage modified with a CAR that targets an antigen is capable of secreting an agent, such as a cytokine or antibody, to aid in macrophage function. Antibodies, such as anti-CD47/antiSIRPα mAB, may also aid in macrophage function. In yet another example, a macrophage modified with a CAR that targets an antigen (such as a protein in a protein aggregate such as α-synuclein or β-amyloid) is engineered to encode a siRNA that aids macrophage function by downregulating inhibitory genes (i.e. SIRPα). Another example, the CAR macrophage is engineered to express a dominant negative (or otherwise mutated) version of a receptor or enzyme that aids in macrophage function.

In some embodiments, a macrophage is modified with multiple genes, wherein at least one gene includes a CAR and at least one other gene comprises a genetic element that enhances CAR macrophage function. In some embodiments, a macrophage is modified with multiple genes, wherein at least one gene includes a CAR and at least one other gene aids or reprograms the function of other immune cells (such as T cells). In some embodiments, a macrophage is modified with multiple genes, wherein at least one gene includes a CAR and at least one other gene comprises a genetic element that enhances therapeutic efficacy. Therapeutic efficacy may be enhanced by a variety of genetic elements including, but not limited to, proteins that act by blocking checkpoint receptors, proteins that have immunostimulatory activity, proteins that have immunosuppressive/anti-inflammatory activity, and proteins that destabilize protein plaques.

Further, in some embodiments, modified cells can be administered to an animal, preferably a mammal, even more preferably a human, to treat a neurodegenerative disease/disorder, an inflammatory disease/disorder, a cardiovascular disease/disorder, a fibrotic disease, amyloidosis or any disease/disorder known in the art to be related to protein misfolding or protein aggregation. In some embodiments, the neurodegenerative disease/disorder comprises tauopathy, α-synucleopathy, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, and prion disease. In addition, in some embodiments, cells of the present invention can be used for the treatment of any condition in which a diminished or otherwise inhibited immune response, especially a cell-mediated immune response, is desirable to treat or alleviate the disease/disorder. In one aspect, the invention includes treating a condition, such as a neurodegenerative disease/disorder, an inflammatory disease/disorder, a cardiovascular disease/disorder, a fibrotic disease, amyloidosis or any disease/disorder known in the art to be related to protein misfolding and/or protein aggregation, in a subject, comprising administering to the subject a therapeutically effective amount of a pharmaceutical composition comprising a population of the cells described herein. In addition, in some embodiments, cells of the present invention can be administered as pre-treatment or conditioning prior to treatment.

In some embodiments, provided cells can also be used to treat inflammatory diseases/disorders that comprises a protein in a protein aggregate, in a tissue of a subject. Examples of such inflammatory diseases/disorders include but are not limited to fibrotic diseases.

In some embodiments, cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease/disorder or condition as determined by those of skill in the art.

In accordance with various embodiments, cells of the invention to be administered may be autologous, allogeneic, xenogeneic, or universal donor with respect to the subject undergoing therapy.

The administration of the cells of the invention may be carried out in any convenient application-appropriate manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i. v.) injection, intraperitoneally, or intracranially. In some embodiments, cells of the invention may be injected directly into a site of inflammation in the subject, a local disease site in the subject, a lymph node, an organ, a tumor, and the like.

### Pharmaceutical Compositions

In some embodiments, pharmaceutical compositions of the present invention may comprise cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease/disorder to be treated (or prevented). The quantity and frequency of administration will be determined by such factors as the condition of the patient, and the type and severity of the patient's disease/disorder, although appropriate dosages may be determined by clinical trials.

When “an immunologically effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the cells described herein may be administered at a dosage of 10⁴ to 10⁹ cells/kg body weight, preferably 10⁵ to 10⁶ cells/kg body weight, including all integer values within those ranges. The cell compositions described herein may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease/disorder and adjusting the treatment accordingly.

In certain embodiments, it may be desired to administer monocytes, macrophages, or dendritic cells to a subject and then subsequently redraw blood (or have an apheresis performed), activate the monocytes, macrophages, or dendritic cells therefrom according to the present invention, and reinfuse the patient with these activated cells. This process can be carried out multiple times every few weeks. In certain embodiments, the cells can be activated from blood draws of from 10 ml to 400 ml. In certain embodiments, the cells are activated from blood draws of 20 ml, 30 ml, 40 ml, 50 ml, 60 ml, 70 ml, 80 ml, 90 ml, or 100 ml. Not to be bound by theory, using this multiple blood draw/multiple reinfusion protocol, may select out certain populations of cells.

In certain embodiments of the present invention, cells are modified using the methods described herein, or other methods known in the art where the cells are expanded to therapeutic levels, are administered to a patient in conjunction with (e.g., before, simultaneously or following) any number of relevant treatment modalities, In an additional embodiment, the cells may be administered before or following a surgery.

The dosage of the above treatments to be administered to a subject will vary with the precise nature of the condition being treated and the recipient of the treatment. The scaling of dosages for human administration can be performed according to art-accepted practices. The dose for CAMPATH antibody, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds/cells of the present invention and practice the claimed methods. The following examples therefore, specifically point out the preferred embodiments of the present invention, and are not to be construed as limiting in any way the remainder of the disclosure.

The materials and methods employed in the below experiments are as described below, unless otherwise specified:

Cell Culture: Cell lines are cultured in RPMI 1640 supplemented with 10% fetal bovine serum and penicillin/streptomycin at 37 C in 5% CO2. The THP-1 monocytic AML cell line is differentiated and induced by culturing cells for 48 hours with 1 ng/mL phorbol 12-myristate 13-acetate in culture media.

Primary Human Macrophages: Primary human monocytes are purified from normal donor apheresis product using Miltenyi CD14 MicroBeads (Miltenyi, 130-050-201). Monocytes are cultured in X-Vivo media supplemented with 5% human AB serum or RPMI 1640 supplemented with 10% fetal bovine serum, with penicillin/streptomycin, glutamax, and 10 ng/mL recombinant human GM-CSF or M-CSF (PeproTech, 300-03) for 7 days in MACS GMP Cell Differentiation Bags (Miltenyi, 170-076-400). Macrophages are harvested on day 5-10 and cryopreserved in FBS+10% DMSO pending subsequent use.

Phagocytosis Assay: Wt or CAR Macrophage red fluorescent (mRFP+) THP1 sublines are differentiated for 48 hours with 1 ng/mL phorbol 12-myristate 13-acetate prior to use in in vitro phagocytosis assays.

Time-Lapse Microscopy: Fluorescent time-lapse video microscopy of CAR mediated phagocytosis is performed using the EVOS FL Auto Cell Imaging System or other digital imaging fluorescent microscope, including a Leica confocal laser scanning microscope (Leica TCS SP8). Images are captured every 1-2 minutes for 6-24 hours. Image analysis is performed with FIJI imaging software and HCS Studio 2.0 Cell Analysis Software (Thermo Fisher).

Lentiviral production and transfection: Chimeric antigen receptor constructs are de novo synthesized by GeneArt (Life Technologies) and cloned into a lentiviral vector as previously described. Concentrated lentivirus is generated using HEK293T cells as previously described. In some instances, a Vpx and accessory plasmid is introduced into the lentiviral packaging process to enhance the transduction efficiency of lentivirus in myeloid cells.

DNA electroporation: In other instances, the CAR is cloned into plasmids of varying length, including minimal length plasmids, and introduced directly into the cell through electroporation, including but not limited to Nucleofection with a Lonza Nucleofector.

Adenoviral production and transfection: Ad5f35 chimeric adenoviral vectors encoding GFP, CAR, or no transgene under a CMV promoter are produced and titrated as per standard molecular biology procedure. Primary human macrophages were transduced with varying multiplicities of infection and serially imaged for GFP expression and viability using the EVOS FL Auto Cell Imaging System. CAR expression is assessed by FACS analysis of surface CAR expression using His-tagged antigen and anti-His-APC secondary antibody (R&D Biosystems Clone AD1.1.10).

Flow Cytometry: FACS was performed on a BD LSR Fortessa. Surface CAR expression is detected with biotinylated protein L (GenScript M00097) and streptavidin APC (BioLegend, #405207) or His-tagged antigen and anti-His-APC secondary antibody (R&D Biosystems Clone AD1.1.10). All flow results are gated on live (Live/Dead Aqua Fixable Dead Cell Stain, Life Technologies L34957) single cells.

RNA Electroporation: CAR constructs are cloned into in vitro transcription plasmids under the control of a T7 promoter using standard molecular biology techniques. CAR mRNA is in vitro transcribed using an mMessage mMachine T7 Ultra In Vitro Transcription Kit (Thermo Fisher), purified using RNEasy RNA Purification Kit (Qiagen), and electroporated into human macrophages using a BTX ECM850 electroporator (BTX Harvard Apparatus). CAR expression is assayed at varying time points post-electroporation using FACS analysis.

Generation of anti-amyloid CAR macrophage: An anti-amyloid CAR was developed using the monoclonal antibody NEOD001 (termed construct 4001). A lentivirus vector containing the mAb NEOD001 CAR construct was used to transduce the acute myeloid leukemia cell line THP-1. One million THP-1 mRFP+ cells were transduced with lentiviruses carrying the 4001 CAR construct at a multiplicity of infection (MOI) of 5:1 (5 viral particles per 1 cell), by incubation in a volume of 2 ml of culture medium (RPMI supplemented with 10% FBS, 1% antibiotics, 1% L-Glutamine, 1% HEPES) at 37° C., for 72 hours. After 72 hours of incubation, cells were washed with culture medium and tested for expression of CAR by flow-cytometry. Biotinylated goat anti-mouse antibody (Biotin-SP-AffiniPure goat anti mouse IgG, F(ab′)2 fragment specific; cat. no 115-065-072-Jackson Immunoresearch) was used for detection of the CAR expression on the surface of THP1 mRFP cells, followed by streptavidin APC (Biolegend 5 μl/stain).

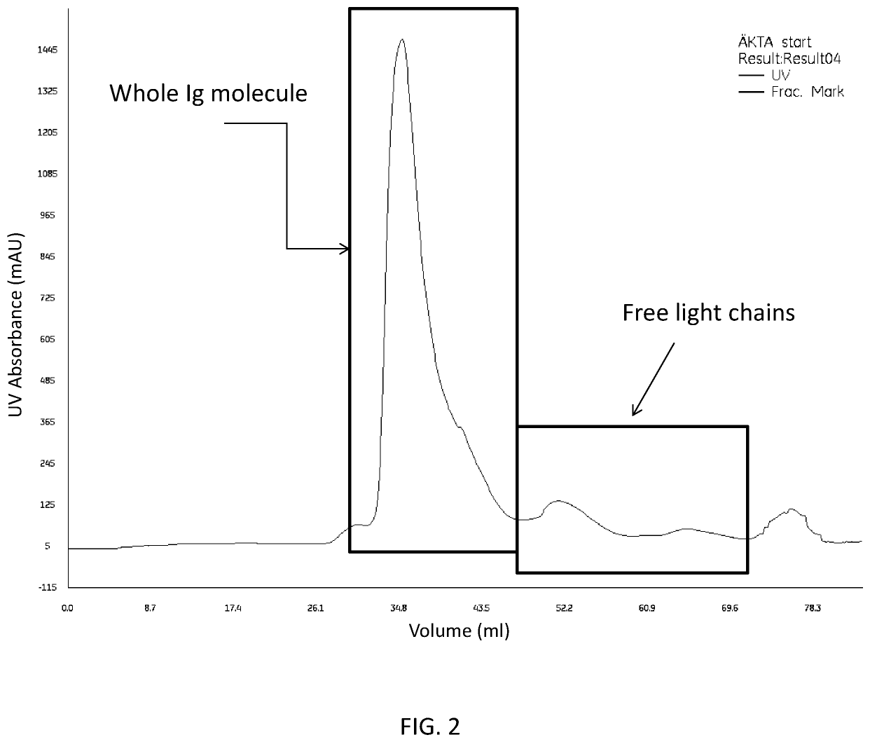

Generation of amyloid fibrils: Amyloid fibrils were obtained by thermal denaturation of immunoglobulin light chains secreted in the cell culture medium by the human cell line AMLC-1 (Arendt B K, Blood. 2008 Sep. 1; 112(5):1931-41). 8-10 million AMLC-1 cells were cultured in T75 flasks, upright, in 40 ml IMDM supplemented with 10% FBS, 1% penicillin/streptomycin, 1% L-Glutamine, and 1 ng/ml human IL-6. The doubling time of this cell line was determined to be 4 days, thus cells were split every 4 days. Secreted light chains were purified from approximately 500 mL of cell culture medium. Before harvesting, cells were grown for 4 days in culture medium without FBS (Opti-MEM). The 500 mL of culture medium were concentrated using Centricon Plus-70 spinning columns (Millipore) with a molecular weight cutoff of 10 kDa, to a final volume of 2 ml. Purification of the free light chains was attained by size-exclusion chromatography, using a Sephadex 75 30/100GL column (GE Healthcare) operated by an AKTA protein purification system. Elution of the Sephadex column was performed using TRIS saline buffer 1 mM, pH=7.2. The chromatography profile is shown in FIG. 2.

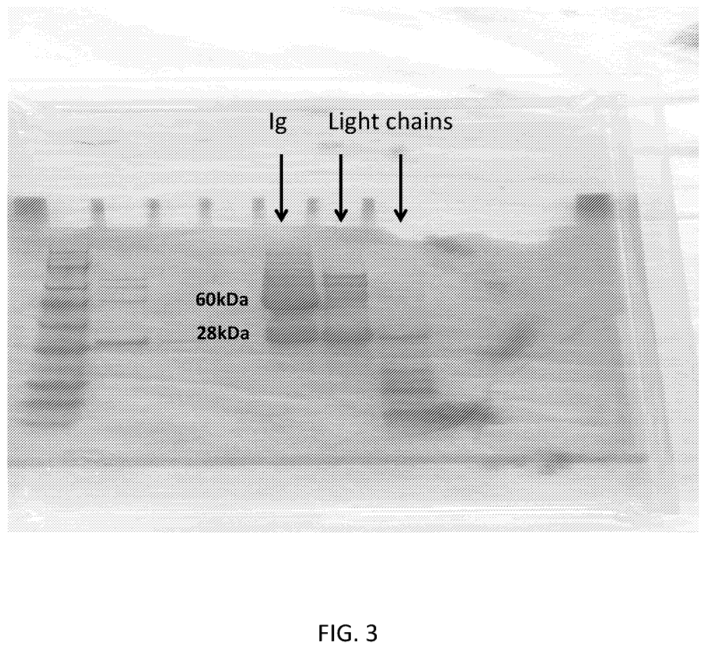

Eluted fractions were concentrated using Amicon spin columns (10 kDa molecular cutoff) to a final volume of 0.5 ml or less and the protein concentration was determined in each fraction using the nanodrop spectrophotometer. The identity of eluted fractions was examined using SDS-PAGE, loading about 20 μg of protein from each fraction in the wells of the gel (FIG. 3). Bolt Novex 4-12% BIS-TRIS PLUS polyacrylmide gels electrophoresis (PAGE) and MES running buffer were used to separate the proteins in each fraction. Free light chains were identified in the 2nd and 3rd fractions eluted from the Sephadex column.

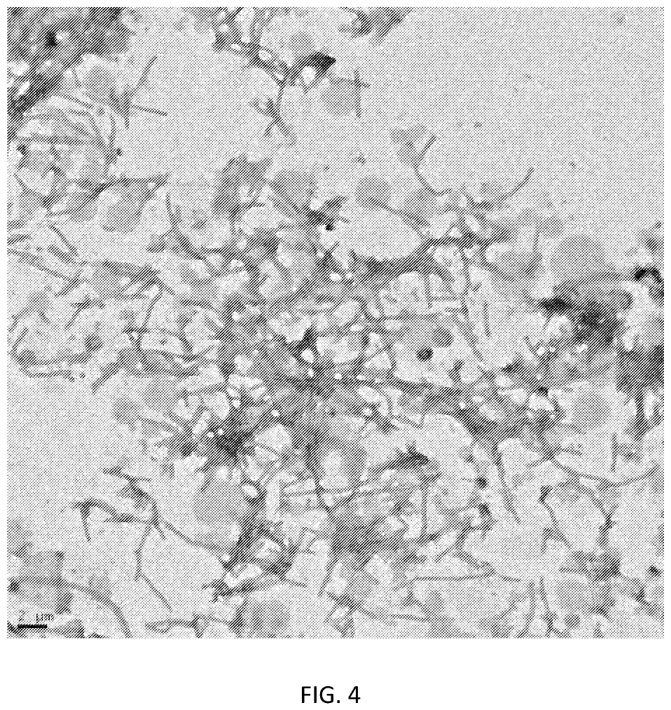

Formation of fibrils of denatured immunoglobulin light chains: In order to obtain fibrils of denatured immunoglobulin light chains, the light chain fraction was thermally denatured at 57.2° C. for 4 days (Arendt B K Blood. 2008 Sep. 1; 112(5):1931-41). The formation of fibrils was assessed by electron microscopy using negative staining (FIG. 4).

Fluorescent labeling of denatured light chains: In vitro testing of the phagocytic function of the differentiated CAR+THP-1mRFP+ cells requires labeling of the denatured fibrils with a fluorescent dye. 100 μg of denatured light chain fibrils were labeled with the fluorescent dye AF488 using the Microscale protein labeling kit (cat. no. A30006, Molecular Probes). The intensity of fluorescent labeling was checked using the nanodrop spectrophotometer.

### Example 1: Generation of CAR Gene-Engineered Macrophages Targeting Serum Amyloid Protein

A CAR construct with an extracellular domain containing the scFv of an antibody recognizing serum amyloid P protein (SAP) is generated by constructing a plasmid containing a 1st generation CAR backbone and the sequence of a commercially available anti amyloid P protein antibody, such as Anti-Serum Amyloid P antibody clone EP1018Y (Abcam catalog number ab45151), clone 14B4 (Abcam catalog number ab27313), clone 5.4A (catalog number CBL304 by Millipore), or any commercially available or proprietary antibody or target recognition moiety that binds to serum amyloid P protein. The NEOD001 scFv that selectively targets a neoepitope on misfolded antibody light chains can also be used. In addition, the extracellular domain of the CAR could comprise a synthetic targeting moiety that recognizes an epitope on SAP, such as an aptamer, a darpin, a naturally occurring or synthetic SAP receptor, an affibody or other engineered protein recognition molecule with affinity towards SAP.

The CAR construct is transfected or transduced into primary human macrophages or a macrophage cell line such as THP-1. Expression of the CAR transcript in the macrophages is evaluated by flow cytometry, demonstrating cell surface expression of the targeting domain.

Targeting of fluorescently-labeled amyloid light chains or of serum amyloid P protein is demonstrated in vitro using a phagocytosis assay and specificity of the targeting interaction between the CAR-macrophages and the target protein is demonstrated by differential phagocytosis of misfolded SAP by comparing activity between control and CAR expressing macrophages. Residual amyloid fibrils are quantified upon removal of the macrophages in order to model residual disease. Active and passive uptake are compared by normalizing the fold enhanced uptake when the assay is performed at 37 degrees Celsius, as compare to 4 degrees Celsius.

### Example 2: Elimination or Reduction of Targets Bearing Serum Amyloid P Protein Epitopes Via Phagocytosis by CAR Gene-Engineered Macrophages

To demonstrate elimination or reduction of the target protein or protein aggregate in an in vivo model, one could use a protein xenograft model, wherein SAP will be conjugated to a fluorescent marker with a direct chemical conjugation kit (AF488 or VivoTrack 680) and surgically implanted into the livers of NSGS mice. Following an engraftment period, mice will receive intravenous injections with either vehicle only (phosphate buffered saline), control non-engineered macrophages, or CAR macrophages directed against SAP (n=5 per group). Injections will be repeated every 3 days for 5 cycles. At the end of treatment, mice will be euthanized and liver tissue will be collected. Whole livers will be imaged for overall fluorescence using an IVIS Spectrum (Perkin Elmer) and the intensity of signal will be compared amongst treatment groups. Hepatic SAP burden will then be quantified by immunohistochemical staining.

Alternatively, elimination or reduction of the target protein or protein aggregate in an in vivo model could be demonstrated in a transgenic mouse model, such as the transgenic mouse expressing human mutant transthyretin (TTR) gene (described in Murakami et al., 1992. Am J Pathol. 1992 August; 141(2): 451-456) or another mouse model where deposition of amyloid is observed.

In the TTR model amyloid deposition begins to be observable at around 6 months of age gradually increasing and eventually detected in multiple organs such as the heart, liver, spleen, stomach, intestine, thyroid gland and/or skin. Once the amyloid deposits are established and detectable by histological analysis or other methods, treatment with CAR macrophages, targeting serum amyloid P protein are conducted by a single tail vein injection of CAR macrophage cells, control CAR macrophages, or control non-engineered macrophages. Reduction in the amyloid deposit is observed by immunohistochemistry of mouse tissues or other methods capable of measuring the amount of amyloid deposit in the mouse organs.

### Example 3: Generation of CAR Gene-Engineered Macrophages Targeting Amyloid Beta

A CAR construct is generated with an extracellular domain containing the scFv of an antibody recognizing amyloid beta, or the tertiary structure of misfolded proteins making up amyloid plaque (Glabe, 2004. Trends Biochem Sci. 2004 October; 29(10):542-7), or oligomer-specific antibodies recognizing misfolded protein forming amyloid plaque (Kayed et al., 2003. Science. April 18; 300(5618):486-9). The CAR is generated by constructing a plasmid containing a 1st generation CAR backbone and the sequence of a commercially available anti amyloid beta antibody (such as ab2539 by Abcam or antibody number 600-401-253S by Rockland Antibodies and Assays), or gammabodies (as described in Perchiacca et al., 2012. PNAS 2012 January, 109 (1) 84-89), or a number of other proprietary or custom synthesized antibodies or other target recognition moieties binding to epitopes on the beta amyloid precursor protein or formed amyloid aggregate.

The CAR construct is transfected or transduced into primary human macrophages or a macrophage cell line such as THP-1 and expression of the CAR transcript in the macrophage cells is evaluated by flow cytometry, which demonstrates cell surface expression of the targeting domain.

Targeting of amyloid precursor protein is demonstrated in vitro via phagocytosis assay and specificity and selectivity of the targeting interaction between the CAR-macrophages and the target protein is demonstrated by differential phagocytosis of antigen-bearing targets as opposed to non-antigen bearing targets. Active and passive uptake are compared by normalizing the fold enhanced uptake when the assay is performed at 37 degrees Celsius, as compare to 4 degrees Celsius. Rescue of neurons from beta amyloid toxicity is modeled by setting up a culture chamber with live human neurons, and adding beta amyloid with or without antigen-specific macrophages. Residual live neurons are quantified microscopically.

### Example 4: Elimination or Reduction of Targets Bearing Amyloid Beta Epitopes Via Phagocytosis by CAR Gene-Engineered Macrophages

To demonstrate elimination or reduction of the target protein or protein aggregate in an in vivo model, one could use a protein xenograft model, wherein beta amyloid protein will be conjugated to a fluorescent marker with a direct chemical conjugation kit (AF488 or VivoTrack 680) and surgically implanted into the livers of NSGS mice. Following an engraftment period, mice will receive intravenous injections with either vehicle only (phosphate buffered saline), control non-engineered macrophages, or CAR macrophages directed against beta amyloid protein (n=5 per group). Injections will be repeated every 3 days for 5 cycles. At the end of treatment, mice will be euthanized and liver tissue will be collected. Whole livers will be imaged for overall fluorescence using an IVIS Spectrum (Perkin Elmer) and the intensity of signal will be compared amongst treatment groups. Hepatic beta amyloid protein burden will then be quantified by immunohistochemical staining.

Alternatively, elimination or reduction of the target protein or protein aggregate in an in vivo model could be demonstrated by using a transgenic mouse model with accumulation of beta amyloid protein in a mouse organ, such as the model with accumulation in the pancreas (Kawarabayashi et al. 1996. Neurobiol Aging 17, 215-222), intestine (Fukuchi et al. 1996. Am J Pathol 149, 219-227) or skeletal muscle (Fukuchi et al. 1998. Am J Pathol 153, 1687-1693) or other mouse model such as the ones reviewed in Philipson et al. 2010. FEBS J: 277(6): 1389-1409 or other models where accumulation of beta amyloid deposits is observed. Once the beta amyloid deposits are established and detectable by histological analysis or other methods, treatment with CAR macrophages, targeting beta amyloid protein are conducted by a single tail vein injection of CAR macrophage cells, or control CAR macrophages, or control non-engineered macrophages. Reduction in the beta amyloid deposit would be observed by immunohistochemistry of the mouse tissues or other methods able to measure the amount of beta amyloid deposit in the mouse organs.

### Example 5: Generation of CAR Gene-Engineered Macrophages Targeting Collagen or Fibrotic Collagen

A CAR construct with an extracellular domain containing the scFv of an antibody recognizing collagen or fibrotic collagen is generated by constructing a plasmid containing a 1st generation CAR backbone and the sequence of a commercially available anti collagen or anti fibrotic collagen antibody or a number of other proprietary or custom synthesized antibodies or other target recognition moieties binding to epitopes on collagen, fibrotic collagen or fibrotic collagen aggregates.

The CAR construct is transfected or transduced into primary human macrophages or a macrophage cell line such as THP-1 and expression of the CAR transcript in the macrophage cells is evaluated by flow cytometry, which demonstrates cell surface expression of the targeting domain.

Targeting of collagen or fibrotic collagen is demonstrated in vitro via phagocytosis assay and specificity and selectivity of the targeting interaction between the CAR-macrophages and the target protein is demonstrated by differential phagocytosis of antigen-bearing targets by comparing CAR macrophages to control macrophages.

### Example 6: Elimination or Reduction of Targets Bearing Collagen Epitopes Via Phagocytosis by CAR Gene-Engineered Macrophages

Fibrotic diseases, or fibrosis, are characterized by pathological deposition of extracellular matrix proteins including collagen. Fibrosis occurs in many organs, notably in the lung (e.g., idiopathic pulmonary fibrosis (IPF)). An IPF model is established after bleomycin challenge in C57BL/6J mice (Limjunyawong et al., 2014. Physiol Rep. February 1; 2(2): e00249). After establishment of IPF, for example after 1, 3, or 6 month post bleomycin administration, the animals receive systemic administration of CAR-macrophages targeting collagen. After 30 days or any other time post bleomycin administration, the animals are sacrificed and lungs are collected for immunohistochemistry and measurement of collagen content via the hydroxyproline assay (Sigma-Aldrich, St. Louis, Mo.).

Alternatively, to demonstrate elimination or reduction of the target protein or protein aggregate in an in vivo model, one could use a protein xenograft model, wherein collagen protein will be conjugated to a fluorescent marker with a direct chemical conjugation kit (AF488 or VivoTrack 680) and surgically implanted into the livers of NSGS mice. Following an engraftment period, mice will receive intravenous injections with either vehicle only (phosphate buffered saline), control non-engineered macrophages, or CAR macrophages directed against collagen protein (n=5 per group). Injections will be repeated every 3 days for 5 cycles. At the end of treatment, mice will be euthanized and liver tissue will be collected. Whole livers will be imaged for overall fluorescence using an IVIS Spectrum (Perkin Elmer) and the intensity of signal will be compared amongst treatment groups. Hepatic collagen protein burden will then be quantified by immunohistochemical staining.

### Example 7: Generation of CAR Gene-Engineered Macrophages Targeting LDL

Atherosclerotic disease leads to increased permeability of the vascular endothelium. Monocytes are drawn into the subendothelial space, where they differentiate into macrophages, which can contribute to the formation of the plaque turning into foam cells. At the same time, the forming plaque retains larger and larger quantities of LDL. A macrophage is engineered to target and phagocytose LDL by constructing a first-generation CAR carrying the scFv of an anti-LDL antibody such as LDL antibody clone 262-01 (catalog number sc-57895, Santa Cruz Biotechnology) or any other commercially available or proprietary antibody or target recognition moiety binding to LDL.

The CAR construct is transfected into primary human macrophages or a macrophage cell line such as THP-1 and expression of the CAR transcript in the macrophage cells is evaluated by flow cytometry, which demonstrates cell surface expression of the targeting domain.

Targeting of LDL is demonstrated in vitro by phagocytosis assay and specificity and selectivity of the targeting interaction between the CAR-macrophages and the target protein is demonstrated by differential phagocytosis of antigen-bearing targets as opposed to non-antigen bearing targets.

### Example 8: Elimination or Reduction of Targets Bearing LDL Epitopes Via Phagocytosis by CAR Gene-Engineered Macrophages

Atherosclerotic disease is recapitulated in one of the well-established mouse atherosclerosis models, such as the ApoE knockout model (Plump et al., 1992. Cell 71:343-353), the LDLR deficient model, other atherosclerosis models (Veseli et al. 2017. Volume 816, 5 Dec. 2017, Pages 3-13), or models that are further modified to enable the experiments described herein. After establishing atherosclerotic disease, the animals receive CAR-macrophages targeting LDL through a tail vein injection and are monitored for a period of time. Upon animal sacrifice, descending thoracic aorta (DA) and/or aortic arch (AA) as well as blood and major organs are collected and analyzed by methods including IHC, RT-PCR, and blood chemistries for levels of atherosclerotic disease.

Alternatively, to demonstrate elimination or reduction of the target protein or protein aggregate in an in vivo model, one could use a protein xenograft model, wherein LDL will be conjugated to a fluorescent marker with a direct chemical conjugation kit (AF488 or VivoTrack 680) and surgically implanted into the livers of NSGS mice. Following an engraftment period, mice will receive intravenous injections with either vehicle only (phosphate buffered saline), control non-engineered macrophages, or CAR macrophages directed against LDL (n=5 per group). Injections will be repeated every 3 days for 5 cycles. At the end of treatment, mice will be euthanized and liver tissue will be collected. Whole livers will be imaged for overall fluorescence using an IVIS Spectrum (Perkin Elmer) and the intensity of signal will be compared amongst treatment groups. Hepatic LDL burden will then be quantified by immunohistochemical staining.

### Example 9: Development of an Anti-Amyloid CAR Macrophage

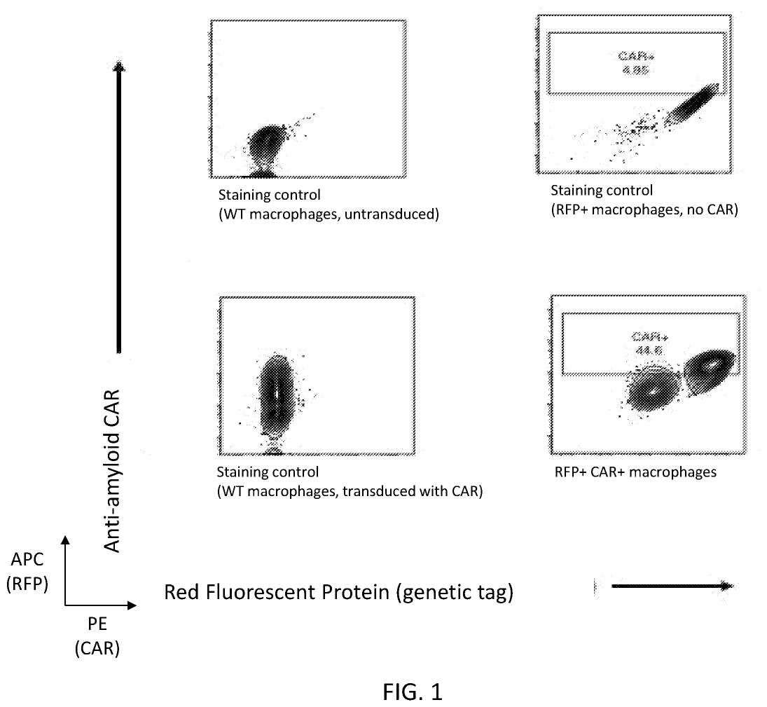

An anti-amyloid CAR macrophage was developed by cloning the sequence of an amyloid-specific scFv into a CAR backbone and transducing cell-line derived macrophages. A representative flow cytometry plot showing expression of the CAR is presented in FIG. 1. The frequency of THP1mRFP+ cells expressing the CAR was determined to be approximately 40%. The cells were sorted and expanded in culture. In order to differentiate the CAR+THP1 cells to macrophages, they were treated with 1 ug/ml PMA and ionomycin for 48-72 h; at the end of this incubation period, cells assumed a macrophage-like morphology.

### Example 10: Clearance of Amyloid Fibrils by Anti-Amyloid Primary Human CAR Macrophages in In Vitro Models

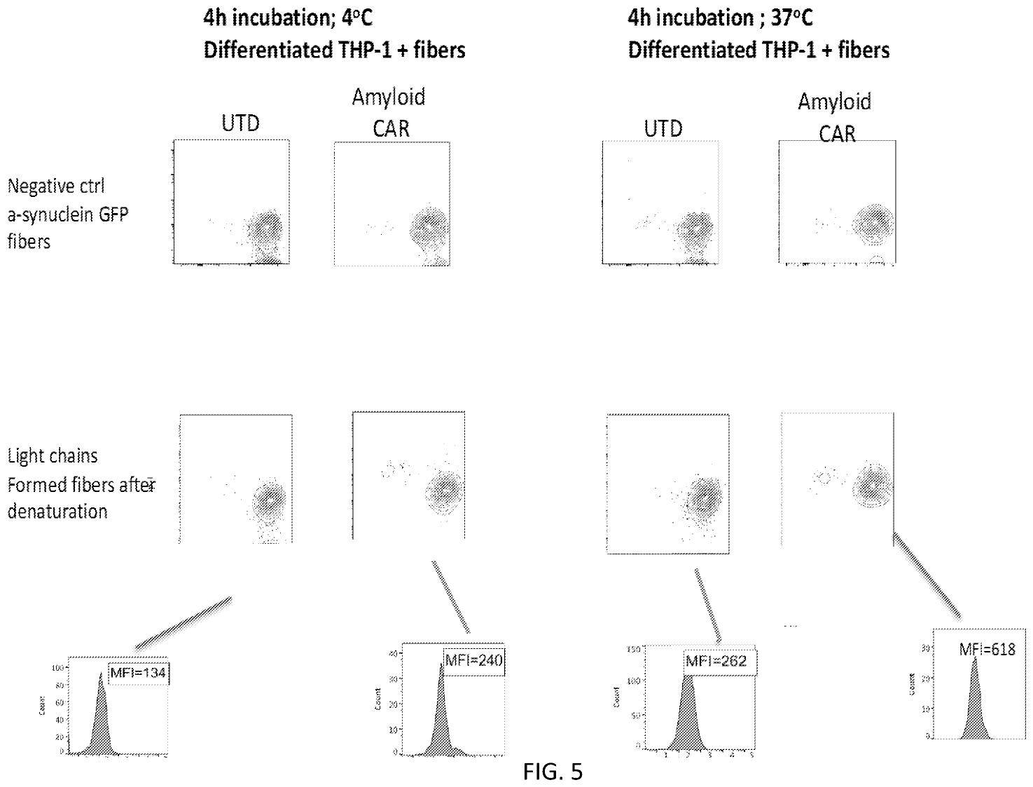

To test the hypothesis that CAR+THP-1mRFP+ cells specifically phagocytose denatured amyloid fibrils, flow-cytometric analysis was performed to detect CAR+THP-1mRFP+ cells that fluoresce in both red (mRFP) and green (AF480) channels. Briefly, 5×10⁴ CAR+THP-1mRFP+ cells or untransduced THP-1mRFP+ cells were incubated at 37° C. for 4h with either AF480 labeled denatured amyloid fibrils, AF480 labeled denatured immunoglobulin protein or with GFP labeled alpha-synuclein fibers as a negative control. To distinguish between phagocytosis and attachment of fibers to the cell surface, a replicate set of tubes was incubated in parallel at 4° C. (since phagocytosis is not expected to occur at 4° C.). Results demonstrated that CAR+THP-1mRFP+ cells did not phagocytose amyloid fibrils at 4° C. (MFI AF480 CAR+THP-1 cells=240; MFI AF480 UTD THP-1 cells=134), whereas at 37° C., the MFI value of AF480 CAR+THP-1 cells doubled (MFI=618) compared to the MFI value of AF480 UTD THP-1 cells (MFI=262), indicating CAR+THP1 cells were able to phagocytose the amyloid fibers (FIG. 5). Further, the specificity of the phagocytosis of the amyloid light chain fibers was represented by the lack of CAR+THP-1mRFP+ cells to phagocytose the GFP+ alpha-synuclein fibrils. In addition, the AF480 labeled denatured immunoglobulin molecules that did not form fibrils showed an attachment to the cell surface rather than phagocytosis, since the CAR+THP-1mRFP+ cells became AF480 positive even when incubated at 4° C.

### OTHER EMBODIMENTS

The recitation of a listing of elements in any definition of a variable herein includes definitions of that variable as any single element or combinations (or subcombinations) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A cell comprising a chimeric antigen receptor (CAR),
wherein the CAR comprises an antigen binding domain, a transmembrane domain and an intracellular domain,
wherein the antigen binding domain is capable of binding to an antigen of a protein aggregate, and
wherein the cell is a monocyte, macrophage and/or a dendritic cell that expresses the CAR.

2. A cell comprising a nucleic acid sequence encoding a chimeric antigen receptor (CAR),
wherein the nucleic acid sequence comprises one or more of a nucleic acid sequence encoding an antigen binding domain, a nucleic acid sequence encoding a transmembrane domain and a nucleic acid sequence encoding an intracellular domain,
wherein the antigen binding domain is capable of binding to an antigen of a protein aggregate, and
wherein the cell is a monocyte, macrophage and/or a dendritic cell that expresses the CAR.

3. The cell of claim 1, wherein the antigen binding domain is capable of binding to an antigen of a protein aggregate in a tissue of a subject with a neurodegenerative disease, an inflammatory disease, a cardiovascular disease, a fibrotic disease or amyloidosis.

4. The cell of claim 1, wherein the intracellular domain is or comprises at least one of a co-stimulatory molecule and a signaling domain.

5. The cell of claim 1, wherein the antigen binding domain is or comprises an antibody agent.

6. The cell of claim 1, wherein the antigen binding domain is or comprises an antibody agent selected from the group consisting of a monoclonal antibody, polyclonal antibody, synthetic antibody, human antibody, humanized antibody, single domain antibody, single chain variable fragment, and antigen-binding fragments thereof.

7. The cell of claim 6, wherein the antibody agent is or comprises a Tau antibody, a TDP-43 antibody, a beta-amyloid antibody, an amyloid antibody, a collagen antibody, and/or an scFV of any of the foregoing antibodies.

8. The cell of claim 3, wherein the neurodegenerative disease is selected from the group consisting of tauopathy, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, Familial British dementia, Fatal Familial Insomnia, Gerstmann-Straussler-Scheinker Syndrome, Hereditary cerebral hemorrhage with amyloidosis (Icelandic) (HCHWA-I), Sporadic Fatal Insomnia (sFI), Variably Protease-Sensitive Prionopathy (VPSPr), Familial Danish dementia, Creutzfeldt-Jakob disease (CJD), Variant Creutzfeldt-Jakob Disease (vCJD), and prion disease.

9. The cell of claim 3, wherein the inflammatory disease is selected from the group consisting of systemic lupus erythematosus, vasculitis, rheumatoid arthritis, periodontitis, ulcerative colitis, sinusitis, asthma, tuberculosis, Crohn's disease, chronic infection, hereditary periodic fevers, malignancies, systemic vasculitides, cystic fibrosis, bronchiectasis, epidermolysis bullosa, cyclic neutropenia, acquired or inherited immunodeficiencies, injection-drug use and acne conglobate, Muckle-Wells (MWS) disease and Familiar Mediterranean Fever (FMF).

10. The cell of claim 3, wherein the amyloidosis is selected from the group consisting of Primary Amyloidosis (AL), Secondary Amyloidosis (AA), Familial Amyloidosis (ATTR), other Familial Amyloidoses, Beta-2 Microglobulin Amyloidosis, Localized Amyloidosis, Heavy Chain Amyloidosis (AH), Light Chain Amyloidosis (AL), Primary Systemic Amyloidosis, ApoAI Amyloidosis, ApoAII Amyloidosis, ApoAIV Amyloidosis, Apolipoprotein C2 Amyloidosis, Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis.

11. The cell of claim 3, wherein the cardiovascular disease is selected from the group consisting of atherosclerosis, coronary artery disease, peripheral artery disease, hypertensive heart disease, metabolic syndrome, hypertension, cerebrovascular disease, and heart failure.

12. The cell of claim 3, wherein the fibrotic disease is selected from the group consisting of pulmonary fibrosis, idiopathic pulmonary fibrosis, cirrhosis, cystic fibrosis, scleroderma, cardiac fibrosis, radiation-induced lung injury, steatohepatitis, glomerulosclerosis, interstitial lung disease, liver fibrosis, mediastinal fibrosis, retroperitoneal cavity fibrosis, bone marrow fibrosis and skin fibrosis.

13. The cell of claim 1, wherein the intracellular domain of the CAR comprises dual signaling domains.

14. The cell of claim 1, wherein an intracellular domain is from a co-stimulatory molecule selected from the group consisting of TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, and any combinations thereof.

15. The cell of claim 1, wherein the intracellular domain is or comprises CD3zeta.

16. The cell of claim 1, wherein the cell exhibits one or more activities selected from the group consisting of phagocytosis, targeted cellular cytotoxicity, antigen presentation, and cytokine secretion.

17. The cell of claim 1, further comprising at least one agent selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate, a lipid, a hormone, a microsome, and any combinations thereof.

18. The cell of claim 1, wherein an activity of the cell is enhanced by inhibition of CD47 and/or SIRPα activity.

19. A pharmaceutical composition comprising a cell of claim 1, and a pharmaceutically acceptable carrier.

20. The pharmaceutical composition of claim 19, further comprising at least one agent selected from the group consisting of a nucleic acid, an antibiotic, an anti-inflammatory agent, an antibody or antibody fragments thereof, a growth factor, a cytokine, an enzyme, a protein, a peptide, a fusion protein, a synthetic molecule, an organic molecule, a carbohydrate, a lipid, a hormone, a microsome, and any combinations thereof.

21. (canceled)

22. A method of treating a neurodegenerative disease, an inflammatory disease, a cardiovascular disease, a fibrotic disease, or amyloidosis, in a subject in need thereof, the method comprising administering to the subject a therapeutically effective amount of a pharmaceutical composition of claim 19.

23. A method for stimulating an immune response to a target cell or tissue in a subject suffering from a neurodegenerative disease, an inflammatory disease, a cardiovascular disease, a fibrotic disease, or amyloidosis, the method comprising administering to a subject a therapeutically effective amount of the pharmaceutical composition of claim 19.

24. A method of modifying a cell, the method comprising introducing into a monocyte, macrophage and/or dendritic cell a chimeric antigen receptor (CAR),
wherein the CAR comprises an antigen binding domain, a transmembrane domain and an intracellular domain,
wherein the antigen binding domain is or comprises an antibody agent capable of binding to an antigen of a protein aggregate.

25. The method of claim 24, wherein introducing the CAR into the cell comprises introducing a nucleic acid sequence encoding the CAR into the cell.

26. The method of claim 25, wherein introducing the nucleic acid sequence into the cell comprises electroporating an mRNA encoding the CAR into the cell.

27. The method of claim 25, wherein introducing the nucleic acid sequence into the cell comprises at least one procedure selected from the group consisting of electroporation, a lentiviral transduction, adenoviral transduction, retroviral transduction and chemical-based transfection.

28. The method of claim 24, wherein the antigen binding domain of the CAR is or comprises an antibody agent selected from the group consisting of a synthetic antibody, human antibody, humanized antibody, single domain antibody, and a single chain variable fragment.

29. The method of claim 24, wherein the antigen binding domain of the CAR is or comprises a Tau antibody, a TDP-43 antibody, a beta-amyloid antibody, an amyloid antibody, a collagen antibody, and/or an scFV of any of the foregoing antibodies.

30. The method of claim 24, wherein the antigen binding domain is capable of binding to an antigen of a protein aggregate in a tissue of a subject with a neurodegenerative disease, an inflammatory disease, a cardiovascular disease, a fibrotic disease or amyloidosis.

31. The method of claim 22, wherein the neurodegenerative disease is selected from the group consisting of tauopathy, presenile dementia, senile dementia, Alzheimer's disease, Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS), Hallervorden-Spatz syndrome, polyglutamine disease, trinucleotide repeat disease, Familial British dementia, Fatal Familial Insomnia, Gerstmann-Straussler-Scheinker Syndrome, Hereditary cerebral hemorrhage with amyloidosis (Icelandic) (HCHWA-I), Sporadic Fatal Insomnia (sF1), Variably Protease-Sensitive Prionopathy (VPSPr), Familial Danish dementia, Creutzfeldt-Jakob disease (CJD), Variant Creutzfeldt-Jakob Disease (vCJD), and prion disease.

32. The method of claim 22, wherein the inflammatory disease is selected from the group consisting of systemic lupus erythematosus, vasculitis, rheumatoid arthritis, periodontitis, ulcerative colitis, sinusitis, asthma, tuberculosis, Crohn's disease, chronic infection, hereditary periodic fevers, malignancies, systemic vasculitides, cystic fibrosis, bronchiectasis, epidermolysis bullosa, cyclic neutropenia, acquired or inherited immunodeficiencies, injection-drug use and acne conglobate, Muckle-Wells (MWS) disease and Familiar Mediterranean Fever (FMF).

33. The method of claim 22, wherein the amyloidosis is selected from the group consisting of Primary Amyloidosis (AL), Secondary Amyloidosis (AA), Familial Amyloidosis (ATTR), other Familial Amyloidoses, Beta-2 Microglobulin Amyloidosis, Localized Amyloidosis, Heavy Chain Amyloidosis (AH), Light Chain Amyloidosis (AL), Primary Systemic Amyloidosis, ApoAI Amyloidosis, ApoAII Amyloidosis, ApoAIV Amyloidosis, Apolipoprotein C2 Amyloidosis, Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis.

34. The method of claim 22, wherein the cardiovascular disease is selected from the group consisting of atherosclerosis, coronary artery disease, peripheral artery disease, hypertensive heart disease, metabolic syndrome, hypertension, cerebrovascular disease, and heart failure.

35. The method of claim 22, wherein the fibrotic disease is selected from the group consisting of pulmonary fibrosis, idiopathic pulmonary fibrosis, cirrhosis, cystic fibrosis, scleroderma, cardiac fibrosis, radiation-induced lung injury, steatohepatitis, glomerulosclerosis, interstitial lung disease, liver fibrosis, mediastinal fibrosis, retroperitoneal cavity fibrosis, bone marrow fibrosis and skin fibrosis.

36. The method of claim 24, further comprising modifying the cell to deliver to a target an agent selected from the group consisting of a nucleic acid, an antibiotic, an anti-inflammatory agent, an antibody, a growth factor, a cytokine, an enzyme, a protein, a peptide, a fusion protein, a synthetic molecule, an organic molecule, a carbohydrate or the like, a lipid, a hormone, a microsome, and any combinations thereof.

37. A composition comprising a cell made by the method of claim 24.
